(19) United States
(12) Patent Application Publication
(10) Pub. No.: US 2011/0281270 A1
(43) Pub. Date: Nov. 17, 2011

(54) EFFICIENT DETECTION OF DOUBLE MUTANTS OF THE CEBPA GENE IN ACUTE MYELOID LEUKEMIA

(75) Inventors: Erik Van Beers, Utrecht (NL); Bas Wouters, Rotterdam (NL); Hendrik Rudolf Delwel, Rotterdam (NL); Peter Jacobus Maria Valk, Rotterdam (NL); Bob Löwenberg, Rotterdam (NL)

(73) Assignee: Erasmus University Medical Center Rotterdam, GE Rotterdam (NL)

(21) Appl. No.: 13/128,483

(22) PCT Filed: Nov. 13, 2009
(86) PCT No.: PCT/EP2009/065178

§ 371 Date: May 26, 2011

(30) Foreign Application Priority Data
Nov. 13, 2008 (EP) 08105794.5

(51) Int. Cl. C12Q 1/68

(52) U.S. Cl. 435/6.12; 435/6.18; 435/6.1

(57) ABSTRACT

The invention is in the field of molecular diagnostics for cancer, in particular, for acute myeloid leukemia (AML). The invention provides methods for diagnosing AML patients with a favorable prognosis. We have found that not all AML patients carrying a CEBPA mutation may have a more favorable prognosis. We found that only the group with double mutations, i.e., biallelic mutations, have a particularly favorable prognosis. We also found a method that distinguishes mono-allelic CEBPA mutations from bi-allelic mutations.

### CROSS-REFERENCE TO RELATED APPLICATIONS

This is a national phase entry under 35 U.S.C. §371 of International Patent Application PCT/EP2009/065178, filed Nov. 13, 2009, published in English as International Patent Publication WO 2010/055147 A1 on May 20, 2010, which claims the benefit under Article 8 of the Patent Cooperation Treaty to European Patent Application Serial No. 08105794.5, filed Nov. 13, 2008.

### TECHNICAL FIELD

The invention is in the field of molecular diagnostics for cancer, in particular, for acute myeloid leukemia (AML). The invention provides methods for diagnosing AML patients with a favorable prognosis.

### BACKGROUND

Acute myeloid leukemia (AML) is not a single disease but a group of neoplasms with various genetic abnormalities and variable responses to treatment. The pretreatment karyotype is still essential in therapy decision-making in AML (Mrozek et al., Blood Rev. 2004, 18:115-136. In recent years, a number of novel molecular markers have been associated with AML prognostics (Mrozek et al., Hematology Am. Soc. Hematol. Educ. Program 2006:169-177; Estey et al., Lancet 2006, 368:1894-1907).

Mutations in the CEBPA gene encoding the CCAAT/enhancer binding protein alpha (C/EBPalpha) are commonly found in AML. Patients carrying monoallelic or biallelic mutations in CEBPA were found to belong to a subgroup with a relatively good prognosis of AML (Barjesteh van Waalwijk et al., Hematol. J. 2003, 4:31-40).

These studies usually require extensive nucleotide sequence analysis for and real-time PCR. There remains a need in the art for alternative detection methods to identify AML patients with a more favorable diagnosis.

### SUMMARY OF THE INVENTION

We have found that not all AML patients carrying a CEBPA mutation may have a more favorable prognosis. We found that only the group with double mutations, i.e., biallelic mutations, have a particularly favorable prognosis. We also found a method that distinguishes mono-allelic CEBPA mutations from bi-allelic mutations.

That method relies on the analysis of the expression level of a set of genes, for instance, in a microarray. The set of genes is detailed in the examples below.

The invention, therefore, relates to a method for determining whether a patient carries a biallelic CEBPA mutation by determining the expression levels of a set of at least two genes selected from the group consisting of the classifier genes as described herein.

Mutations in CCAAT/enhancer binding protein alpha (CEBPA) are found in 5-10% of acute myeloid leukemia (AML) and have been associated with a favorable clinical outcome. The majority of AMLs with CEBPA mutations simultaneously carries two mutations, which are usually biallelic (CEBPA^{double-mut}), while other AMLs only carry a single heterozygous mutation (CEBPA^{single-mut}). Here, we identified, using denaturing high performance liquid chromatography and nucleotide sequencing, 41 CEBPA mutant cases in a cohort of 598 newly diagnosed AMLs, i.e., 28 CEBPA^{double-mut} cases and 13 CEBPA^{single-mut} cases. Genome-wide gene expression profiling and clinical outcome analysis revealed that CEBPA^{double-mut} AMLs associated with a unique gene expression profile and a favorable outcome. In contrast, CEBPA^{single-mut} AMLs did not express a discriminating signature, and could not be distinguished from wild-type cases with regard to clinical outcome. These results demonstrate significant underlying heterogeneity within CEBPA mutation positive AML with important implications for assessment of prognosis.

Mutations in the transcription factor CCAAT/enhancer binding protein alpha (CEBPA) are found in 5-10% of acute myeloid leukemia (AML).¹⁻⁹ CEBPA mutations have been associated with a relatively favorable outcome and have, therefore, gained interest as a promising novel prognostic marker.^{3,4,9,10} While variable sequence variations have been described, two prototypical classes of mutations are most frequent. N-terminal mutations are located between the major translational start codon and a second ATG in the same open reading frame. These mutations introduce a premature stop of translation of the p42 CEBPA protein and increased translation of a p30 isoform that may inhibit the function of full length protein.⁶ Mutations in the C-terminal basic leucine zipper (bZIP) region, in contrast, are in-frame, and may impair dimerization and/or DNA binding.⁷ Remaining mutations in CEBPA are found between the N-terminus and bZIP region.

The majority CEBPA mutant AML carries two mutations. Most frequently, this is a combination of an N-teiiiiinal and a bZIP mutation.^{7,8,11} In AMLs with two CEBPA mutations, the mutations are usually on different alleles, hence no wild-type CEBPA protein is expressed. A similar condition is found in cases carrying a homozygous mutation. However, there are also AMLs that only have one single heterozygous mutation, and thus retain expression of a wild-type allele.

To obtain better insight into the exact distribution of the various types of CEBPA mutations in de novo adult AML and into their impact on clinical outcome, we have studied a cohort of 598 cases. Using denaturing high performance liquid chromatography (dHPLC) followed by nucleotide sequencing, we identified cases with two different mutations or one homozygous mutation (further referred to as double mutations; CEBPA^{double-mut}) as well as cases with only one single heterozygous mutation (CEBPA^{single-mut}). Genome-wide gene expression profiling (GEP) revealed that CEBPA^{double-mut} AMLs expressed a highly characteristic signature, while CEBPA^{single-mut} cases did not. More unexpectedly, a favorable prognostic effect was uniquely associated with double mutations. These results reveal the presence of unknown heterogeneity within AML with CEBPA mutations that may have important implications for clinical prognostication.

In a cohort of 598 cases of adult de novo AML, we identified 65 cases with an aberrant profile in at least one of the three investigated amplicons of the CEBPA coding sequence (FIG. 1, Panels A and B). The presence of a CEBPA sequence variation was confirmed by nucleotide sequencing. Cases that only carried an insertion polymorphism^{11,14-16} or variation(s) that did not lead to amino acid changes were considered wild-type. Two additional specimens were not considered in further analysis because they carried in-frame variations of unknown significance outside the bZIP region. As a result, 41/598 CEBPA^{mut} AML cases (6.9%) were considered. These included 13 CEBPA ^{single-mut} cases and 28 CEBPA^{double-mut} cases, i.e., AMLs with either homozygous or two distinct mutations (Table 2). Additional screening of the remaining 547 AML cases using a combination of agarose gel analysis and nucleotide sequencing as described³ did not reveal mutations that had been missed by dHPLC.

We found that the expression level of genes selected from a group of 25 genes was highly predictive for the occurrence of CEBPA double mutants. Every combination of two genes, selected from the group of 25 genes shown in Table 5, was found to predict the occurrence of a CEBPA double mutation to an acceptable level. The sensitivity and specificity of the method improved when the expression levels of more than two genes were determined, such as 3, 4, 5, 6, 7, 8, 9, 10, 11, 12, 13, 14, 15, 16, 17, 18, 19, 20, 21, 22, 23, 24 or 25 genes.

In a preferred embodiment of the invention, the two genes are selected from the group of genes shown in Table 11.

In a particularly preferred method according to the invention, the expression of at least two genes selected from a set of seven genes shown in Table 12 is determined, preferably the expression level of 3, 4, 5, 6 or 7 genes selected from the group of genes shown in Table 12 is determined.

In another particularly preferred method according to the invention, the expression of at least two genes selected from a set of nine genes shown in Table 13 is determined, preferably the expression level of 3, 4, 5, 6, 7, 8 or 9 genes selected from the group of genes shown in Table 13 is determined.

Best results were obtained when the expression level of all seven genes from Table 12 or all nine genes from Table 13 were determined.

The expression level of the genes identified herein may be determined in various ways known in the art. Particularly preferred is the use of specific probe sets as identified herein. Exemplary useful probe sets are provided in the accompanying sequence listing. Other probe sets may be designed by the skilled person based on the primary sequence of the genes identified herein, which are available from various public sources.

In a further preferred method, a prescreening is performed wherein the level of expression of the CEBPA gene is determined and compared to a predetermined value. If the expression level of the CEBPA gene in a particular sample is above that predetermined value, then the above methods may be performed on those samples and this combination assay then provides even more reliable results. The examples provide a method for reliably determining the predetermined value.

In the terminology used herein, a gene is identified and characterized in that it encodes an expression product comprising a nucleic acid sequence that is capable of specifically hybridizing, preferably under high stringency conditions, to the cDNA sequences provided in the sequence listings. Preferably, the genes encode an expression product that is more than 90% homologous to the sequences provided in the sequence listing, such as 92, 94, 96, 97, 98, 99 or even 100% homologous.

Table 10 provides details of the genes as described herein, reference to public databases is made that will allow the skilled person to unequivocally determine the identity and sequence of the particular genes.

The skilled person will be aware of the definition of high stringency conditions, further guidance is to be obtained from Sambrook et al., Molecular Cloning: A Laboratory Manual third edition.

The skilled person will also be aware of the fact that many splice variants of the genes mentioned herein may exist and he is well capable of designing specific primers and probes for such splice variants if necessary.

### BRIEF DESCRIPTION OF THE DRAWINGS

FIG. 1: Panel A: Schematic representation of the CEBPA gene and location of PCR primers used for dHPLC analysis of fragments a, b and c. Functional regions are depicted, i.e., two transactivation domains (TAD1 and TAD2) in the N-terminal part, and the basic leucine zipper (bZIP) region in the C-terminal part. Nucleotide (nt) position is indicated relative to the main translation start site. Amino acid (aa) numbering and the alternative translation start site at position nt 358 (aa 120) are also depicted. Panel B: Representative profiles of dHPLC analysis of one of the three investigated fragments, i.e., amplicons b, in a random selection of 90 samples. Heteroduplexes (various colors) are released earlier than homoduplexes (green), and can, therefore, be recognized as distinct peaks. Time is depicted on the x-axis, and voltage on the y-axis. Panel C: A gene expression prediction signature for CEBPA mutations (irrespective of single or double mutant status) was derived in a data set of 524 AMLs, including 38 CEBPA^{mut} cases. Prediction accuracy for each of the 38 CEBPA^{mut} cases was estimated using repeated ten-fold cross-validation as detailed herein. The proportion of correct predictions for the selected 38 CEBPA mutant specimens is indicated (upper panel). The heat map in the lower panel depicts the resulting 19 probe set gene expression classifier, comprising a good discriminating signature for CEBPA^{mut} AML (see Table 6 for probe set infomiation). Intensity values (log2) were mean centered over the cohort of 524 AML cases. For visualization purposes, the genes were hierarchically clustered (Euclidian distance, average linkage). Cells represent relative log2 expression values, and were color coded on a scale ranging from bright green (−3) to bright red (+3). Panel D: Kaplan Meier curves showing difference in OS between CEBPA^{mut} and CEBPA^{wt} AML, log rank test P=0.027. Panel E: Differences in OS between CEBPA^{double-mut} versus CEBPA^{wt} AML, P=0.004, and versus CEBPA^{single-mut} AML, P=0.005. Panel F: Restricted analysis to patients younger than 60: differences in OS between CEBPA^{double-mut} versus CEBPA^{wt} AML, P=0.0096, and versus CEBPA^{single-mut} AML, P=0.033. Panel G: Restricted analysis to patients with normal cytogenetics: differences in OS between CEBPA^{double-mut} versus CEBPA^{wt} AML, P=0.069, and versus CEBPA^{single-mut} AML, P=0.024.

FIG. 2: Principal component analysis of GEP data based on 19-probe set prediction signature for CEBPA mutations. Principal component analysis of 524 cases of AML was carried out based on the 19 probe sets that constitute the prediction signature for CEBPA mutation irrespective of single or double mutant status (Table 6). Each square represents an AML case. AMLs were color coded based on CEBPA status: CEBPA^{double-mut} (red), CEBPA^{single-mut} (blue) and CEBPA^{wt} (yellow). Cases belonging to a previously described subgroup of myeloid/T-lymphoid leukemias characterized by epigenetic silencing of CEBPA have been colored in green. The first two principal components (PCA1 and PCA2) have been depicted. The figure illustrates that CEBPA^{double-mut} can be completely separated from CEBPA^{wt} cases over the first principal component (PCA1), while the CEBPA^{single-mut} cases are scattered within the wild-type cohort. In addition to CEBPA^{double-mut,} there are also some other AMLs that are clearly separated from the wild-type cohort—these all represent CEBPA silenced AMLs.

FIG. 3: Kaplan Meier curves for event-free survival. Panel A: Kaplan Meier curves showing difference in EFS between CEBPA^{mut} and CEBPA^{wt} AML, log rank test P=0.050. Panel B: Differences in EFS between CEBPA^{double-mut} versus CEBPA^{wt} AML, P=0.005, and versus CEBPA ^{single-mut} AML, P=0.004. Panel C: Restricted analysis to patients younger than 60: differences in EFS between CEBPA^{double-mut} versus CEBPA^{wt} AML, P=0.014, and versus CEBPA^{single-mut} AML, P=0.026. Panel D: Restricted analysis to patients with normal cytogenetics: differences in EFS between CEBPA^{double-mut} versus CEBPA^{wt} AML, P=0.081, and versus CEBPA^{single-mut} AML, P=0.093.

FIG. 4: Procedure for a preferred method according to the invention wherein sample is tested for the elevated expression of the CEBPA gene and if found above a predetermined value, a set of genes is tested from the groups described herein.

FIG. 5: Determining the predetermined value for a method as depicted in FIG. 4.

FIG. 6: Unsupervised hierarchical cluster analysis of the expression values of the seven classifier genes shown in Table 12. Clustering CEBPA double mutant versus non-double mutant; seven genes used to cluster the samples (cosine—complete).

FIG. 7: Principal component analysis plot of the data obtained in FIG. 6.

FIG. 8: Unsupervised hierarchical cluster analysis of the expression values of the nine classifier genes shown in Table 13. Clustering CEBPA double mutant versus non-double mutant; nine genes used to cluster the samples (cosine—complete).

FIG. 9: Principal component analysis plot of the data obtained in FIG. 8.

TABLE 1
Clinical and molecular data

| | AML | | AML | |
|---|---|---|---|---|
| | cohort1 | | cohort2 | |
| | (n = 247) | | (n = 214) | |
| Gender | | | | |
| Male | 119 | | 113 | |
| Female | 128 | | 101 | |
| Age (median (range)) | 43 | (15-60) | 46 | (17-60) |
| White blood cell count | 30 | (0-278) | 29 | (1-349) |
| Bone marrow blast count | 68 | (0-98) | 64 | (0-96) |
| Platelet count | 49 | (3-931) | 59 | (5-998) |
| FAB | | | | |
| M0 | 6 | | 10 | |
| M1 | 55 | | 41 | |
| M2 | 54 | | 52 | |
| M3 | 17 | | 7 | |
| M4 | 43 | | 41 | |
| M5 | 62 | | 42 | |
| M6 | 3 | | 3 | |
| not determined | 7 | | 18 | |
| Cytogenetics* | | | | |
| normal | 99 | (41%) | 95 | (46%) |
| inv(16) | 21 | (9%) | 16 | (7%) |
| t(15; 17)** | 18 | (7%) | 7 | (3%) |
| t(8; 21) | 21 | (9%) | 14 | (7%) |
| t(6; 9) | 4 | (2%) | 2 | (1%) |
| abn3q | 7 | (3%) | 9 | (4%) |
| del5(q) | 3 | (1%) | 12 | (6%) |
| del7(q) | 17 | (7%) | 14 | (7%) |
| 11q23 | 13 | (5%) | 8 | (4%) |
| +8 | 22 | (9%) | 11 | (5%) |
| t(9; 22) | 4 | (2%) | 1 | (<1%) |
| complex | 13 | (5%) | 21 | (10%) |
| other | 63 | (26%) | 45 | (22%) |
| Mutations* | | | | |
| CEBPA | 16 | (6%) | 15 | (7%) |
| NPM1 | 77 | (31%) | 63 | (29%) |
| FLT3-ITD | 65 | (26%) | 61 | (29%) |
| FLT3-TKD | 30 | (12%) | 19 | (9%) |
| KRAS | 4 | (2%) | 0 | (0%) |
| NRAS | 23 | (9%) | 22 | (10%) |

TABLE 2
Details of identified CEBPA mutations, Detected
mutations/sequence variations

| | | |
|---|---|---|
| N-terminus before 2^{nd} | | |
| ATG | bZIP | other |

Double mutants

| | | | |
|---|---|---|---|
| 1316 | del357C | 1106-1107ins3bp | — |
| 2169 | del213C | 1066-1067ins18 | — |
| 2192 | ins396GG | 1060-1062dup | — |
| 2230 | del381C | 1076-1078dup | — |
| 2234 | 392-395dup | 1084-1089dup | — |
| 2240 | del332C | 1076-1078dup | — |
| 2242 | del252-261 | dup1064-1066 | — |
| 2253 | del213 | 1051-1052ins36 | — |
| 2273 | ins472T | 1062-1079dup | — |
| 2748 | ins424A | 1057-1058ins3bp | — |
| 2753 | del302-317 | 1085-1087dup | — |
| 3117 | 397delC | 1090insAAG | — |
| 6735 | del377-389 | 1087insCAG | — |
| 6975 | 397C > T (stop) | 1076insTGG | — |
| 7127 | 437delG | 1090insAAG | — |
| 7142 | 354delCG | 1090insAAG | — |
| 7148 | 248delTT | 1072insGTGGAGACGCAGC | — |
| | | ACCTAAAATCG | |
| 7149 | 382delC | 1090insAAG | — |
| 7406 | 406delCGGCAGC | 1087insCAG | — |
| 2218 | — | dup1062-1094 (hom.) | — |
| 3101 | ins362CC (hom.) | — | — |
| 3327 | — | 1104-1115del (hom.) | — |
| 6376 | — | 1091insTGCTGGAGCTGCAG | — |
| | | CGCAACGTGGAGACGCAG | |
| | | CAGAAGG (hom.) | |
| 2545 | 474C > G (stop) | — | 813delG |
| 4336 | 219insC | 1016G > C (R > P)* | — |
| 5362 | 349delTACATCGACCC | 1033G > C, A > P* | — |
| 5352 | 311delG | — | 678ins13bp |
| 5364 | 376insG | — | 486-522dup |

Single mutants

| | | | |
|---|---|---|---|
| 2176 | 218insC | — | — |
| 2194 | ins468AACC | — | — |
| 4341 | 309 CGG > TT | — | — |
| 6462 | 445insCCAA | — | — |
| 7075 | 219insC | — | — |
| 7302 | 505delG | — | — |
| 2188 | — | — | 852insCCCGC |
| | | | AC |
| 6247 | | — | 564insTA |
| 6362 | — | — | 648insG |
| 2237 | — | 1188-1189ins125 | — |
| 3096 | — | 1114-1134del | — |
| 7185 | — | 1029insGGACCC | — |
| 7324 | — | 1075-1203dup | — |

Other (not included in analysis)

| | | | |
|---|---|---|---|
| 2183 | — | — | 722-736dup |
| 5359 | — | — | 575C > G, R > T |

*Point mutations in basic leucine zipper region, located in highly conserved amino acid. Vinson C. R., P. B. Sigler, and S. L. McKnight. Scissors-grip model for DNA recognition by a family of leucine zipper proteins. Science 1989, 246: 911-916.

TABLE 3
Clinical and molecular characteristics of 524 AML cases included
in survival analysis

| | N | Median (Range) |
|---|---|---|
| Sex | | |
| Male | 263 | |
| Female | 261 | |
| Age | | 46.5 (15.0-77.0) |
| Younger than 60 | 460 | |
| Older than 60 | 64 | |
| WBC × 10\hat{ }9/L | | 28.2 (0.3-510) |
| Blasts in BM % | | 65 (0-98) |
| Platelets × 10\hat{ }9/L | | 55 (3-998) |
| FAB* | | |
| M0 | 18 | |
| M1 | 105 | |
| M2 | 127 | |
| M3 | 25 | |
| M4 | 92 | |
| M5 | 114 | |
| M6 | 7 | |
| RAEB-t | 10 | |
| 10 ( . . . ) | 4 | |
| 11 ( . . . ) | 19 | |
| Cytogenetic risk group | | |
| Good | 103 | |
| Intermediate | 305 | |
| Poor | 104 | |
| Cytogenetics^{†} | | |
| Normal cytogenetics | 214 | |
| t(15; 17) | 25 | |
| t(8; 21) | 37 | |
| Inv(16) | 41 | |
| 11q23 | 22 | |
| 3q | 20 | |
| −5(q) | 16 | |
| −7(q) | 37 | |
| Chromosome 8 | 37 | |
| Complex karyotype | 33 | |
| Other cytogenetic abnormality | 130 | |
| Molecular abnormalities | | |
| FLT3-ITD | 141 | |
| FLT3-TKD | 53 | |
| NPM1 | 157 | |
| NRAS | 50 | |
| KRAS | 5 | |

*FAB indicates French-American-British classification
^{†}Each cytogenetic abnormality was taken into account, irrespective of the presence of other cytogenetic abnormalities.

TABLE 4
Clinical and molecular characteristics of CEBPA^{single-mut} and
CEBPA^{double-mut} AML cases

| | CEBPA^{double-mut} | | CEBPA^{single-mut} | | |
|---|---|---|---|---|---|
| | cases | | cases | | |
| | (N = 26) | | (N = 12) | | |
| | | Median | | Median | |
| | N | (Range) | N | (Range) | P* |
| Sex | | | | | 0.85 |
| Male | 16 | | 7 | | |
| Female | 10 | | 5 | | |
| Age | | 45 (16-75) | | 52 (20-70) | 0.27 |
| Age group | | | | | 0.40 |
| Younger than 60 | 24 | | 10 | | |
| Older than 60 | 2 | | 2 | | |
| WBC × 10\hat{ }9/L | | 35.7 (3.4-174) | | 11.4 (2.8-263.4) | 0.65 |
| Blasts in BM % | | 65 (25-94) | | 70 (16-92) | 0.66 |
| Platelets × | | 51 (8-265) | | 89 (18-174) | 0.087 |
| 10\hat{ }9/L | | | | | |
| FAB^{†} | | | | | 0.24 |
| M0 | 0 | | 0 | | |
| M1 | 15 | | 3 | | |
| M2 | 7 | | 4 | | |
| M3 | 0 | | 0 | | |
| M4 | 2 | | 3 | | |
| M5 | 1 | | 0 | | |
| M6 | 0 | | 0 | | |
| RAEB-t | 0 | | 1 | | |
| 10 ( . . . ) | 0 | | 0 | | |
| 11 ( . . . ) | 1 | | 1 | | |
| Cytogenetic | | | | | 0.56 |
| risk group | | | | | |
| Good | 0 | | 0 | | |
| Intermediate | 25 | | 11 | | |
| Poor | 1 | | 1 | | |
| Cytogenetics** | | | | | |
| Normal | 20 | | 7 | | 0.24 |
| cytogenetics | | | | | |
| t(15; 17) | 0 | | 0 | | NA |
| t(8; 21) | 0 | | 0 | | NA |
| Inv(16) | 0 | | 0 | | NA |
| 11q23 | 0 | | 0 | | NA |
| 3q | 0 | | 0 | | NA |
| −5(q) | 1 | | 0 | | 0.49 |
| −7(q) | 1 | | 1 | | 0.56 |
| Chromosome 8 | 0 | | 2 | | 0.03* |
| Complex | 0 | | 1 | | 0.14 |
| karyotype | | | | | |
| Other cytogenetic | 5 | | 2 | | 0.85 |
| abnormality | | | | | |
| Molecular | | | | | |
| abnormalities | | | | | |
| FLT3-ITD | 3 | | 5 | | 0.034* |
| FLT3-TKD | 0 | | 2 | | 0.033* |
| NPM1 | 0 | | 3 | | 0.0079* |
| NRAS | 3 | | 1 | | 0.76 |
| KRAS | 0 | | 0 | | NA |

P values for chi-square test (categorical variables) and Mann-Whitney test (continuous variables) are given.
*P value < 0.05.
^{†}FAB indicates French-American-British classification.
NA: not applicable.
**Each cytogenetic abnormality was taken into account, irrespective of the presence of other cytogenetic abnormalities

TABLE 5
25-probe set signature for CEBPA double mutations.

| | Probe Set ID | Gene Symbol | Score 1* | Score 2* |
|---|---|---|---|---|
| 1 | 222423_at | NDFIP1 | 0.0365 | −0.6993 |
| 2 | 1555630_a_at | RAB34 | 0.031 | −0.5929 |
| 3 | 211682_x_at | UGT2B28 | −0.0278 | 0.5323 |
| 4 | 217800_s_at | NDFIP1 | 0.0271 | −0.519 |
| 5 | 223095_at | MARVELD1 | 0.0225 | −0.4307 |
| 6 | 202252_at | RAB13 | 0.0182 | −0.3482 |
| 7 | 1553183_at | UMODL1 | −0.0108 | 0.2071 |
| 8 | 217853_at | TNS3 | 0.0093 | −0.1783 |
| 9 | 1554300_a_at | LOC136306 | −0.009 | 0.1733 |
| 10 | 224710_at | RAB34 | 0.0078 | −0.1487 |
| 11 | 201841_s_at | HSPB1 /// MEIS3 | 0.0077 | −0.1483 |
| 12 | 222422_s_at | NDFIP1 | 0.0077 | −0.1473 |
| 13 | 234247_at | — | −0.0069 | 0.132 |
| 14 | 227423_at | LRRC28 | −0.0057 | 0.1091 |
| 15 | 200765_x_at | CTNNA1 | 0.0056 | −0.1075 |
| 16 | 217226_s_at | SFXN3 | 0.0056 | −0.1072 |
| 17 | 220393_at | GLULD1 | −0.0045 | 0.086 |
| 18 | 224822_at | DLC1 | −0.0024 | 0.046 |
| 19 | 220974_x_at | SFXN3 | 0.0011 | −0.0204 |
| 20 | 215772_x_at | SUCLG2 | 9.00E−04 | −0.0165 |
| 21 | 206726_at | PGDS | −8.00E−04 | 0.0162 |
| 22 | 232227_at | HSPC324 | | |
| 23 | 1553183_at | TUBB6 | | |
| 24 | 1556599_s_at | ARPP-21 | | |
| 25 | 204039_at | CEBPA | | |

*Shrunken centroids for class 1 (CEBPA^{wt}) and class 2 (CEBPA^{double-mut}), respectively.

TABLE 6
19-probe set signature for CEBPA mutation.

| | Probe Set ID | Gene symbol | Score 1* | Score 2* |
|---|---|---|---|---|
| 1 | 222423_at | NDFIP1 | 0.0228 | −0.291 |
| 2 | 223095_at | MARVELD1 | 0.0209 | −0.2676 |
| 3 | 211682_x_at | UGT2B28 | −0.0188 | 0.241 |
| 4 | 1555630_a_at | RAB34 | 0.0172 | −0.2202 |
| 5 | 201841_s_at | HSPB1 /// MEIS3 | 0.0166 | −0.2127 |
| 6 | 217800_s_at | NDFIP1 | 0.0135 | −0.1726 |
| 7 | 215772_x_at | SUCLG2 | 0.0123 | −0.1575 |
| 8 | 202252_at | RAB13 | 0.0114 | −0.1462 |
| 9 | 220974_x_at | SFXN3 | 0.01 | −0.1283 |
| 10 | 217226_s_at | SFXN3 | 0.0094 | −0.1209 |
| 11 | 217853_at | TNS3 | 0.0094 | −0.1201 |
| 12 | 212459_x_at | SUCLG2 | 0.0075 | −0.0958 |
| 13 | 1553183_at | UMODL1 | −0.0069 | 0.0877 |
| 14 | 227423_at | LRRC28 | −0.0055 | 0.071 |
| 15 | 1554300_a_at | LOC136306 | −0.0055 | 0.0704 |
| 16 | 200765_x_at | CTNNA1 | 0.0039 | −0.0504 |
| 17 | 227845_s_at | SHD | −0.0017 | 0.0217 |
| 18 | 204039_at | CEBPA | −6.00E−04 | 0.008 |
| 19 | 224822_at | DLC1 | −2.00E−04 | 0.0029 |

*Shrunken centroids for class 1 (CEBPA^{wt}) and class 2 (CEBPA^{mut}), respectively.

TABLE 7
Multivariable Cox's proportion hazards models.

| | OS | | EFS | |
|---|---|---|---|---|
| Total cohort (N = 524) | HR (95% CI) | P* | HR (95% CI) | P* |
| A. | | | | |
| CEBPA^{mut †} | 0.48 (0.29-0.78) | 0.0035* | 0.53 (0.34-0.83) | 0.0053* |
| Intermediate^{‡} | 2.15 (1.48-3.14) | <0.001* | 1.96 (1.40-2.75) | <0.001* |
| Poor^{‡} | 3.29 (2.23-4.85) | <0.001* | 2.79 (1.96-3.98) | <0.001* |
| Age [decades] | 1.18 (1.08-1.28) | <0.001* | 1.10 (1.02-1.19) | 0.012* |
| WBC^{§} | 1.30 (1.02-1.64) | 0.032* | 1.23 (0.98-1.54) | 0.07 |
| FLT3-ITD^{\|} | 1.58 (1.22-2.07) | <0.001* | 1.51 (1.17-1.94) | 0.0014* |
| NPM1^{\|} | 0.57 (0.43-0.76) | <0.001* | 0.54 (0.41-0.72) | <0.001* |
| B. | | | | |
| CEBPA^{single-mut †} | 1.18 (0.58-2.40) | 0.65 | 1.61 (0.82-3.17) | 0.16 |
| CEBPA^{double-mut †} | 0.32 (0.17-0.61) | <0.001* | 0.35 (0.20-0.62) | <0.001* |
| Intermediate^{‡} | 2.21 (1.52-3.22) | <0.001* | 2.05 (1.46-2.87) | <0.001* |
| Poor^{‡} | 3.35 (2.27-4.94) | <0.001* | 2.85 (2.00-4.06) | <0.001* |
| Age [decades] | 1.17 (1.08-1.28) | <0.001* | 1.10 (1.02-1.19) | 0.014* |
| WBC^{§} | 1.33 (1.05-1.68) | <0.019* | 1.29 (1.03-1.62) | 0.025* |
| FLT3-ITD^{\|} | 1.56 (1.20-2.03) | <0.001* | 1.46 (1.14-1.89) | 0.0031* |
| NPM1^{\|} | 0.55 (0.41-0.74) | <0.001* | 0.51 (0.39-0.67) | <0.001* |
| C. Younger than 60 | | | | |
| CEBPA^{single-mut †} | 1.08 (0.48-2.45) | 0.85 | 1.55 (0.72-3.33) | 0.26 |
| CEBPA^{double-mut †} | 0.31 (0.15-0.61) | <0.001* | 0.34 (0.19-0.62) | <0.001* |
| Intermediate^{‡} | 2.50 (1.67-3.74) | <0.001* | 2.24 (1.57-3.21) | <0.001* |
| Poor^{‡} | 3.74 (2.48-5.65) | <0.001* | 3.06 (2.11-4.43) | <0.001* |
| Age [decades] | 1.13 (1.02-1.26) | 0.019* | 1.09 (0.99-1.20) | 0.093 |
| WBC^{§} | 1.35 (1.04-1.75) | 0.024* | 1.31 (1.03-1.67) | 0.029* |
| FLT3-ITD^{\|} | 1.59 (1.19-2.11) | 0.0014* | 1.44 (1.10-1.89) | 0.0088* |
| NPM1^{\|} | 0.50 (0.37-0.69) | <0.001* | 0.47 (0.35-0.63) | <0.001* |
| D. Normal | | | | |
| cytogenetics | | | | |
| CEBPA^{single-mut †} | 1.81 (0.72-4.56) | 0.21 | 1.46 (0.57-3.61) | 0.44 |
| CEBPA^{double-mut ‡} | 0.43 (0.22-0.86) | 0.017* | 0.45 (0.24-0.84) | 0.012* |
| Age [decades] | 1.16 (1.01-1.32) | 0.035* | 1.06 (0.94-1.20) | 0.33 |
| WBC^{§} | 1.83 (1.22-2.74) | 0.0035* | 1.40 (0.96-2.04) | 0.079 |
| FLT3-ITD^{\|} | 1.59 (1.09-2.32) | 0.016* | 1.57 (1.10-2.25) | 0.012* |
| NPM1^{\|} | 0.47 (0.32-0.70) | <0.001* | 0.48 (0.33-0.70) | <0.001* |

HR indicates hazard ratio,
CI indicates confidence interval,
ITD indicates internal tandem duplication.
*P value <0.05
^{†}CEBPA status versus CEBPA^{wt}
^{‡}Cytogenetic risk versus cytogenetic good risk
^{§}White blood cell count higher than 20 × 10\hat{ }9/L versus lower than 20 × 10\hat{ }9/L
^{|}FLT3-ITD versus no FLT3-ITD
^{|}NPM1 mutation versus no NPM1 mutation

TABLE 8
Identification of CEBPA gene

| | |
|---|---|
| Affymetrix ID | 204039_at |
| Gene Name | CEBPA |
| SEQ ID NO: | 144 |
| Ensembl Gene | ENSG00000184771 |
| UniGene | Hs.76171 |
| EntrezGene | 1050 |
| Swiss Prot | P49715, Q6P3S4 |
| GenBank | BX381916 BM454745 BX343831 BG166894 BX343822 |
| Accession | BX357445 BG481789 BF343807 BG480539 AW387104 |
| | BP381812 BM685384 BP318479 BP316640 BU536048 |
| | BE901458 DB355660 BE301008 AI971171 N39553 |
| | AI133307 BF897933 AA974969 BQ320594 BE817700 |
| | AI335709 H25130 AI359788 W87364 |
| Clone ID | CS0DI071YP19 IMAGE: 5583238 CS0DI026YB17 |
| | IMAGE: 4455116 CS0DI025YB17 IMAGE: 4652008 |
| | IMAGE: 4151059 IMAGE: 4652989 adSE00431 |
| | UI-E-CI0-aac-b-01-0-UI PCD07787 PCD01895 |
| | IMAGE: 6563891 IMAGE: 3957506 |
| | HEMBA1002093 IMAGE: 2906902 |
| | IMAGE: 2488780 IMAGE: 243975 IMAGE: 1555532 |
| Ensembl* Chr | 19 |
| Start (bp) | 38484084 |
| End (bp) | 38485044 |
| Strand | −1 |
| CEBPA | >0.9295 |
| Double | |
| Mutant | |
| non-CEBPA | <=0.9295 |
| Double | |
| Mutant | |

TABLE 9
Identification of the genes in a classifier according to a preferred embodiment

| | Affymetrix ID | | | | | |
|---|---|---|---|---|---|---|
| | | 200765_s_at | | | | |
| | 223095_at | 200764_s_at | 1555630_a_at | 232227_at | 1553183_at | 209191_at |
| Gene Name | MARVELD1 | CTNNA1 | RAB34 | HSPC324 mRNA | UMODL1 | TUBB6 |
| SEQ ID NO | 145 | 146 and 147 | 148 | 149 | 150 | 151 |
| Ensembl Gene | ENSG00000155254 | ENSG00000044115 | ENSG00000109113 | | ENSG00000177398 | ENSG00000176014 |
| UniGene | Hs.209614 | Hs.534797 | Hs.301853 | | Hs.242520 | Hs.193491 |
| EntrezGene | | 1495 | 83871 | | 89766 | 84617 |
| SwissProt | Q9BSK0 | B3KSR8, | A8MYQ9, | | Q5DID0, | B3KS31 |
| | | B4DKT9, | B4DNC0, | | Q6L9N1, | Q2NKY5 |
| | | B4DU00, | B4E3A0, | | Q6L9N2, | Q9BUF5 |
| | | B4E2G8, | Q6NXT3, | | Q6L9N3, | |
| | | P35221, | Q96PJ7, | | Q6L9N4, | |
| | | Q8N1C0 | Q9BZG1 | | Q6L9N5, | |
| | | | | | Q6L9N6, | |
| | | | | | Q6L9N7, | |
| | | | | | Q6L9N8, | |
| | | | | | Q6L9N9 | |
| GenBank | AF325259 | NM_001903 | NM_031934 | | NM_173568 | AK092677 |
| Accession | AK025996 | U03100 | AK074689 | | AY771619 | BC002654 |
| | CR596454 | L23805 | CR596043 | | AB051810 | NM_032525 |
| | AK027093 | D14705 | CR622817 | | NM_001004416 | CR616368 |
| | BE893438 | BC000385 | CR613380 | | AY771620 | AK001295 |
| | CB987999 | D13866 | CR612137 | | AB051811 | AK098440 |
| | BQ069359 | AK094151 | CR620011 | | AK124223 | AK022340 |
| | BM467964 | BC031262 | BC016841 | | AK093654 | CR605380 |
| | BI760667 | AK022326 | CR614283 | | AK093909 | CR595733 |
| | CB998043 | AK055751 | CR600820 | | AB096971 | CR607214 |
| | AL561889 | BM471580 | BM923740 | | AB096970 | CR593961 |
| | BF689317 | BG325862 | CR612854 | | AB096969 | CR593665 |
| | BQ680528 | BM470327 | BM908364 | | AB096967 | BC111374 |
| | BQ678428 | BM541985 | BM543785 | | AB096968 | CR594370 |
| | BX427275 | BQ921033 | BC066904 | | BC039531 | CR614714 |
| | BG680011 | BG331611 | AJ277106 | | BM554176 | BQ931764 |
| | BQ688723 | BM911471 | AK027312 | | AB096966 | CD518467 |
| | BX427274 | CR591756 | BC091510 | | AB096972 | BM459986 |
| | BU146020 | BM910414 | BM927206 | | DB119958 | BQ051130 |
| | BQ685706 | BE872885 | BI160806 | | DB203306 | BM808494 |
| | BF525741 | BF308274 | BQ688695 | | AB096973 | BM473459 |
| | BE784598 | BI115155 | BM471195 | | DB120482 | BM916088 |
| | BQ431733 | BQ680232 | BG678790 | | DB105484 | AL570105 |
| | BG119599 | BG421344 | AL572680 | | DA667096 | BM916277 |
| | BU557883 | BG324521 | BQ690161 | | DB342848 | BQ050656 |
| | BQ685699 | BM809837 | BM542780 | | DB107494 | BE562844 |
| | AL520554 | AL517624 | BM459933 | | AI674725 | BE908682 |
| | BQ722260 | BU174894 | AL582669 AL | | DB34454 | BM916793 AL |
| | BU8 | BM549588 B | | | | |
| Clone ID | IMAGE: 3921691 | IMAGE: 5517928 | IMAGE: 5759466 | | IMAGE: 5742385 | IMAGE: 6464655 |
| | IMAGE: 30340385 | IMAGE: 4562616 | IMAGE: 5430926 | | THYMU2027857 | IMAGE: 30396260 |
| | IMAGE: 5811757 | IMAGE: 5539050 | IMAGE: 5589119 | | TRACH2014018 | IMAGE: 5532341 |
| | IMAGE: 5532864 | IMAGE: 5527347 | IMAGE: 5768168 | | THYMU2028637 | IMAGE: 5785949 |
| | IMAGE: 5185189 | IMAGE: 6470427 | IMAGE: 5018808 | | THYMU2006277 | IMAGE: 5471125 |
| | IMAGE: 30348746 | IMAGE: 4550616 | IMAGE: 6251210 | | NCRRP2000438 | IMAGE: 5561909 |
| | | | | | THYMU2009363 | |
| | CS0B006YF24 | IMAGE: 5466258 | IMAGE: 5517701 | | IMAGE: 2328624 | IMAGE: 5482386 |
| | IMAGE: 4299175 | IMAGE: 5453895 | IMAGE: 4749251 | | THYMU3000028 | CS0DI006YL18 |
| | IMAGE: 6273218 | IMAGE: 3854669 | CS0DI033YF23 | | THYMU2035004 | IMAGE: 5482619 |
| | IMAGE: 6257558 | IMAGE: 4131441 | IMAGE: 6206810 | | IMAGE: 2686446 | IMAGE: 5784634 |
| | CS0DM009YB15 | IMAGE: 5022066 | IMAGE: 5520781 | | IMAGE: 1369237 | IMAGE: 3690396 |
| | IMAGE: 4751839 | IMAGE: 6264343 | IMAGE: 5532285 | | IMAGE: 2714078 | IMAGE: 3901613 |
| | IMAGE: 6247732 | IMAGE: 4590040 | CS0DL007YP05 | | IMAGE: 3220570 | IMAGE: 5483264 |
| | IMAGE: 6182685 | IMAGE: 4560593 | CS0DC027YN21 | | IMAGE: 3210444 | IMAGE: 5480342 |
| | IMAGE: 6250286 | IMAGE: 5454742 | CS0DF036YK16 | | | CS0DI048YB17 |
| | IMAGE: 4212835 | CS0DA003YN06 | IMAGE: 5431145 | | | IMAGE: 5573184 |
| | IMAGE: 3877109 | IMAGE: 6253265 | IMAGE: 30335877 | | | CS0DF032YF04 |
| | IMAGE: 61594040 | IMAGE: 5441502 | IMAGE: 6255765 | | | IMAGE: 5812025 |
| | IMA | IMAG | IMAGE: | | | IMAGE |
| Ensembl* Chr | 10 | 5 | 17 | | 21 | 18 |
| Start (bp) | 99463469 | 138117006 | 24065429 | | 42356137 | 12298215 |
| End (bp) | 99467897 | 138298619 | 24069328 | | 42436174 | 12316551 |
| Strand | 1 | 1 | −1 | | 1 | 1 |
| CEBPA | −1.2350042 | −1.1971764 | −1.1888662 | 1.1646809 | 1.1563656 | −1.1314407 |
| Double Mutant | | | | | | |
| non-CEBPA | 0.6058511 | 0.5872941 | 0.5832174 | −0.5713529 | −0.5672737 | 0.5550464 |
| Double Mutant | | | | | | |

TABLE 10
Identification of the genes in a classifier according to a preferred embodiment

| | Affymetrix ID | | | |
|---|---|---|---|---|
| | 1555630_a_at | 211682_x_at | 223095_at | 202252_at |
| Gene Name | RAB34 | UGT2B10 | MARVELD1 | RAB13 |
| SEQ ID NO: | 148 | 152 | 145 | 153 |
| Ensembl Gene | ENSG00000109113 | ENSG00000109181 | ENSG00000155254 | ENSG00000143545 |
| UniGene | Hs.301853 | Hs.201634 | Hs.209614 | |
| EntrezGene | 83871 | 7365 | | 5872 |
| | A8MYQ9,B4 | B5BUM0, | Q9BSK0 | P51153 |
| | DNC0, | Q9BY64 | | Q504R6 |
| | B4E3A0, | | | |
| | Q6NXT3, | | | |
| | Q96PJ7, | | | |
| | Q9BZG1 | | | |
| GenBank | NM_031934 | AK292738 | AF325259 | |
| Accession | AK074689 | BC I 13649 | AK025996 | |
| | CR596043 | AK222872 | CR596454 | |
| | CR622817 | AK222839 | AK027093 | |
| | CR613380 | NM_001075 | BE893438 | |
| | CR612137 | CD013995 | CB987999 | |
| | CR620011 | CD013994 | BQ069359 | |
| | BC016841 | BG208263 | BM467964 | |
| | CR614283 | BG189133 | BI760667 | |
| | CR600820 | BG460626 | CB998043 | |
| | BM923740 | DA636871 | AL561889 | |
| | CR612854 | BG207749 | BF689317 | |
| | BM908364 | BG 193837 | BQ680528 | |
| | BM543785 | BG212458 | BQ678428 | |
| | BC066904 | BG201991 | BX427275 | |
| | AJ277106 | CD610479 | BG680011 | |
| | AK027312 | DV080704 | BQ688723 | |
| | BC091510 | DV080703 | BX427274 | |
| | BM927206 | AV657530 | BU146020 | |
| | BI160806 | CB113377 | BQ685706 | |
| | BQ688695 | BJ994179 | BF525741 | |
| | BM471195 | BP239062 | BE784598 | |
| | BG678790 | BJ996532 | BQ431733 | |
| | AL572680 | DA035754 | BG119599 | |
| | BQ690161 | DA036946 | BU557883 | |
| | BM542780 | CB161428 | BQ685699 | |
| | BM459933 | CB164598 | AL520554 | |
| | AL582669 AL | DA035976 | BQ722260 | |
| | | BJ | BU8 | |
| Clone ID | IMAGE: 5759466 | LIVER1000008 | IMAGE: 3921691 | |
| | IMAGE: 5430926 | MGC9.4.1.1.1.D12 | IMAGE: 30340385 | |
| | IMAGE: 5589119 | GLCFCH04 | IMAGE: 5811757 | |
| | IMAGE: 5768681 | L7N800102-1-G03 | IMAGE: 5532864 | |
| | | hmfn-2739 | | |
| | IMAGE: 5018808 | HEP05515 | IMAGE: 5185189 | |
| | | hyst-1724 | | |
| | IMAGE: 6251210 | BEAST2001235 | IMAGE: 30348746 | |
| | IMAGE: 5517701 | BEAST2002650 | CS0DB006YF24 | |
| | IMAGE: 4749251 | L 1 7N670205 | IMAGE: 4299175 | |
| | | n1-44-E11 | | |
| | CS0DI033YF23 | L17N670205 | IMAGE: 6273218 | |
| | | n1-40-B03 | | |
| | IMAGE: 6206810 | BEAST2001491 | IMAGE: 6257558 | |
| | IMAGE: 5520781 | hmfn-1058 | CS0DM009YB15 | |
| | IMAGE: 5532285 | LIVER2000454 | IMAGE: 4751839 | |
| | CS0DL007YP05 | BRHIP3026232 | IMAGE: 6247732 | |
| | CS0DC027YN21 | LIVER2005346 | IMAGE: 6182685 | |
| | CS0DF036YK16 | TLIVE2000399 | IMAGE: 6250286 | |
| | IMAGE: 5431145 | BEAST1000079 | IMAGE: 4212835 | |
| | IMAGE: 30335877 | LIVER2001765 | IMAGE: 3877109 | |
| | IMAGE: 6255765 | FEKI | IMAGE: 6159404 | |
| | IMAGE: | | IMA | |
| Ensembl* Chr | 17 | 4 | 10 | 1 |
| Start (bp) | 24065429 | 69716300 | 99463469 | 152220756 |
| End (bp) | 24069328 | 69731217 | 99467897 | 152225430 |
| Strand | −1 | 1 | 1 | −1 |
| CEBPA | −1.1888662 | 2.5589241 | −1.2350042 | −2.5430716 |
| Double Mutant | | | | |
| non-CEBPA | 0.5832174 | −0.1719174 | 0.6058511 | 0.1708524 |
| Double Mutant | | | | |
| | Affymetrix ID | | | |
| | | 200765_x_at | | |
| | 1556599_s_at | 200764s_att | 222423_at | 217853_at |
| Gene Name | ARPP-21 | CTNNA1 | | TNS3 |
| SEQ ID NO: | 154 | 146 and 147 | 155 | 156 |
| Ensembl Gene | | ENSG00000044115 | ENSG00000131507 | ENSG00000136205 |
| UniGene | | Hs.534797 | Hs.698981 | Hs.520814 |
| EntrezGene | 100130503, | 1495 | 80762 | 64759 |
| | 10777 | | | |
| | A8K0T4, | B3KSR8, | B2RDB8, | B2RNV1 , |
| | A8KIF3, | B4DKT9, | Q9BT67 | Q59GW4, |
| | B4DG96, | B4DU00, | | Q68CZ2, |
| | Q9UBL0 | B4E2G8, | | Q8NDL6, |
| | | P35221, | | Q9H8D5 |
| | | Q8N1C0 | | |
| GenBank | | NM_001903 | | NM_022748 |
| Accession | | U03100 | | CR749644 |
| | | L23805 | | AF378756 |
| | | D14705 | | AB208995 |
| | | BC000385 | | BX648770 |
| | | D13866 | | BX649002 |
| | | AK094151 | | AB062750 |
| | | BC031262 | | AF417489 |
| | | AK022326 | | AK023794 |
| | | AK055751 | | AL833845 |
| | | BM471580 | | AK001539 |
| | | BG325862 | | AL137468 |
| | | BM470327 | | AK092864 |
| | | BM541985 | | AK002151 |
| | | BQ921033 | | CR612439 |
| | | BG331611 | | CR626304 |
| | | BM911471 | | BC071791 |
| | | CR591756 | | BM549238 |
| | | BM910414 | | BM464033 |
| | | BE872885 | | BM479835 |
| | | BF308274 | | CF455895 |
| | | BI115155 | | BU177971 |
| | | BQ680232 | | BQ219649 |
| | | BG421344 | | AL574224 |
| | | BG324521 | | BX402420 |
| | | BM809837 | | BX343887 |
| | | AL517624 | | BM456641 |
| | | BU174894 | | AL550191 BF |
| | | BM549588 B | | |
| Clone ID | | IMAGE: 5517928 | | IMAGE: 5548500 |
| | | | | IMAGE: 5540062 |
| | | IMAGE: 4562616 | | IMAGE: 5577335 |
| | | | | IMAGE: 30515668 |
| | | IMAGE: 5539050 | | IMAGE: 6110724 |
| | | | | IMAGE: 6052747 |
| | | IMAGE: 5527347 | | CS0DI039YB04 |
| | | | | CS0DI033YA01 |
| | | IMAGE: 6470427 | | IMAGE: 5496318 |
| | | | | IMAGE: 4067904 |
| | | IMAGE: 4550616 | | IMAGE: 4375295 |
| | | | | IMAGE: 6182151 |
| | | IMAGE: 5466258 | | IMAGE: 6183810 |
| | | | | IMAGE: 6021041 |
| | | IMAGE: 5453895 | | IMAGE: 4642284 |
| | | | | IMAGE: 6182344 |
| | | IMAGE: 3854669 | | IMAGE: 6180126 |
| | | | | IMAGE: 6598436 |
| | | IMAGE: 4131441 | | IMAG |
| | | IMAGE: 5022066 | | |
| | | IMAGE: 6264343 | | |
| | | IMAGE: 4590040 | | |
| | | IMAGE: 4560593 | | |
| | | IMAGE: 5454742 | | |
| | | CS0DA003YN06 | | |
| | | IMAGE: 6253265 | | |
| | | IMAGE: 5441502 | | |
| | | IMAG | | |
| Ensembl* Chr | | 5 | 5 | 7 |
| Start (bp) | | 138117006 | 141468508 | 47281277 |
| End (bp) | | 138298619 | 141513222 | 47588216 |
| Strand | | 1 | 1 | −1 |
| CEBPA | 2.4642698 | −1.1971764 | −2.4427875 | −2.3294763 |
| Double Mutant | | | | |
| non-CEBPA | −0.1655582 | 0.5872941 | 0.1641149 | 0.1565023 |
| Double Mutant | | | | |

TABLE 11
Twelve gene probe sets

| Nr from Table 5 | Probe Set ID | Gene Symbol | SEQ ID NO: s |
|---|---|---|---|
| 1 | 222423_at | NDFIP1 | 122-132 |
| | 217800_s_at | | |
| 2 | 1555630_a_at | RAB34 | 45-55 |
| 3 | 211682_x_at | UGT2B28/B10 | 89-99 |
| 5 | 223095_at | MARVELD1 | 12-22 |
| 6 | 202252_at | RAB13 | 100-110 |
| 7 | 1553183_at | UMODL1 | 67-77 |
| 8 | 217853_at | TNS3 | 133/143 |
| 15 | 200765_x_at | CTNNA1 | 23-33 |
| | 200764_x_at | | 34-44 |
| 22 | 232227_at | HSPC324 | 56-66 |
| 23 | 209191_at | TUBB6 | 78-88 |
| 24 | 1556599_s_at | ARPP-21 | 111-121 |
| 25 | 204039_at | CEBPA | 1-11 |

TABLE 12
Seven gene probe sets

| Nr from Table 5 | Probe Set ID | Gene Symbol |
|---|---|---|
| 2 | 1555630_a_at | RAB34 |
| 5 | 223095_at | MARVELD1 |
| 7 | 1553183_at | UMODL1 |
| 15 | 200765_x_at | CTNNA1 |
| | 200764_x_at | |
| 22 | 232227_at | HSPC324 |
| 23 | 1553183_at | TUBB6 |
| 25 | 204039_at | CEBPA |

TABLE 13
Nine gene probe set

| | Probe Set ID | Gene Symbol |
|---|---|---|
| 1 | 222423_at | NDFIP1 |
| | 217800_s_at | |
| 2 | 1555630_a_at | RAB34 |
| 3 | 211682_x_at | UGT2B28/B10 |
| 5 | 223095_at | MARVELD1 |
| 6 | 202252_at | RAB13 |
| 8 | 217853_at | TNS3 |
| 15 | 200765_x_at | CTNNA1 |
| 24 | 1556599_s_at | ARPP-21 |
| 25 | 204039_at | CEBPA |

### EXAMPLES

### AML Samples, mRNA Isolation, dHPLC Analysis and Nucleotide Sequencing

Bone marrow aspirates or peripheral blood samples of 598 cases of de novo AML were collected, blast cells were purified, and mRNA was isolated as reported.¹² The entire CEBPA coding region was investigated by dHPLC and nucleotide sequencing. For details on patient characteristics and experimental procedures, see below.

### Statistical Analysis

Survival was estimated according to the method by Kaplan and Meier. The log rank test was used to assess statistical significance. Multivariable analysis was performed using Cox's proportional hazards models. Definitions for outcome parameters and cytogenetic risk groups have been described.¹³ Further details are given below.

### Gene Expression Profiling Analysis

Gene expression profiles were obtained using Affymetrix HGU133Plus2.0 GeneChips.¹² Details on data processing and analysis are given below.

### Materials and Methods

### Patient Characteristics and Molecular Analyses

CEBPA mutations were assessed in a cohort of 598 cases of de novo AML. Detailed clinical and molecular characteristics were available for 524/598 cases (Table 3). These 524 were enrolled in the Dutch-Belgian Hemato-Oncology Cooperative Group (HOVON)-04, -10, -12, -29, -32, -42, or -43 protocols (available at http://www.hovon.nl). Reverse-transcription polymerase chain reaction (RT-PCR) and sequence analyses for FLT3-ITD, FLT3-TKD, NPMJ, N-RAS, and K-RAS, mutations were performed as described previously.¹⁻³

### Detection of CEBPA Mutations

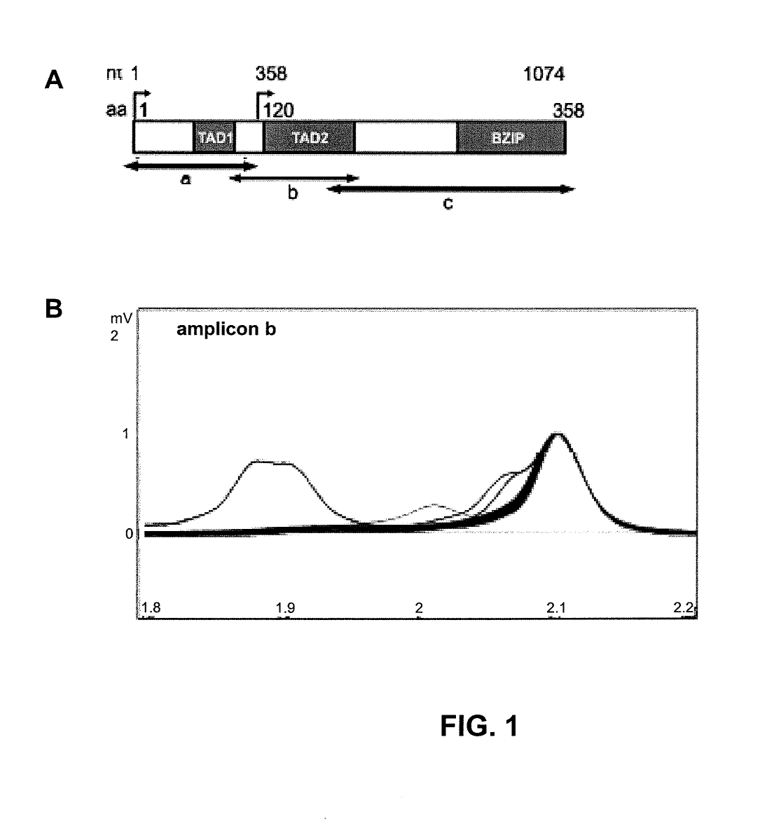

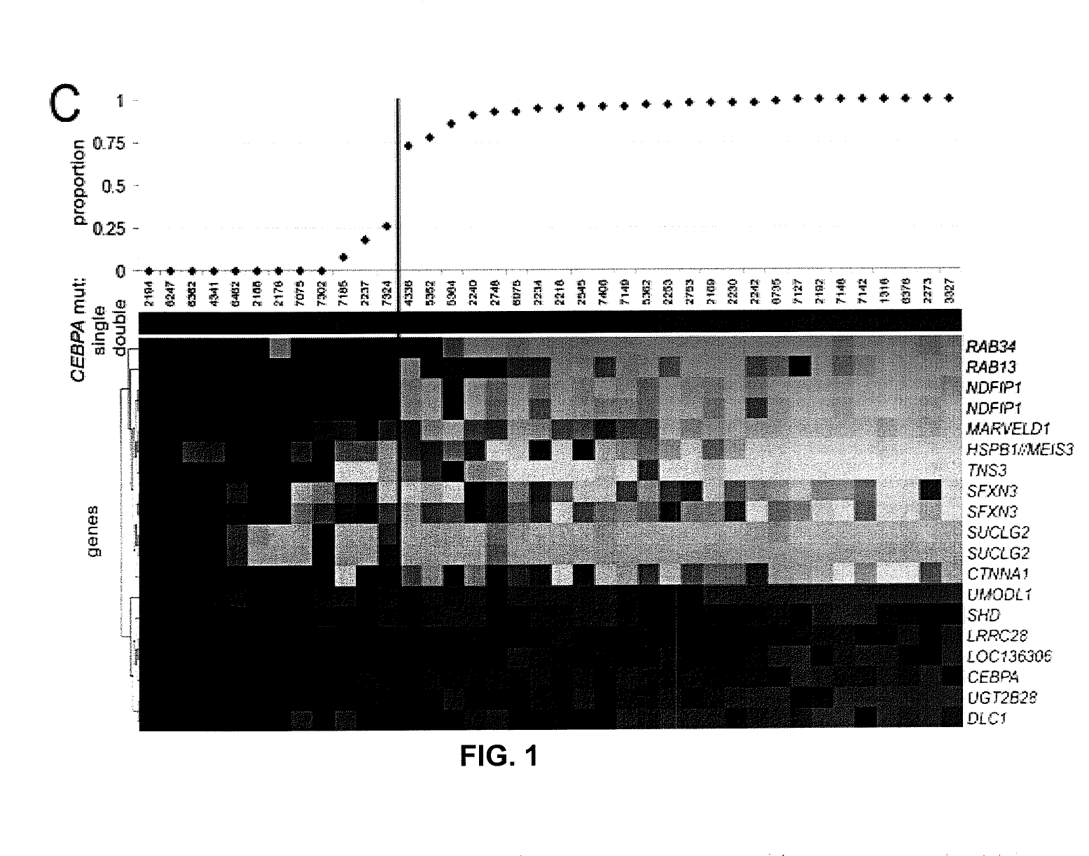

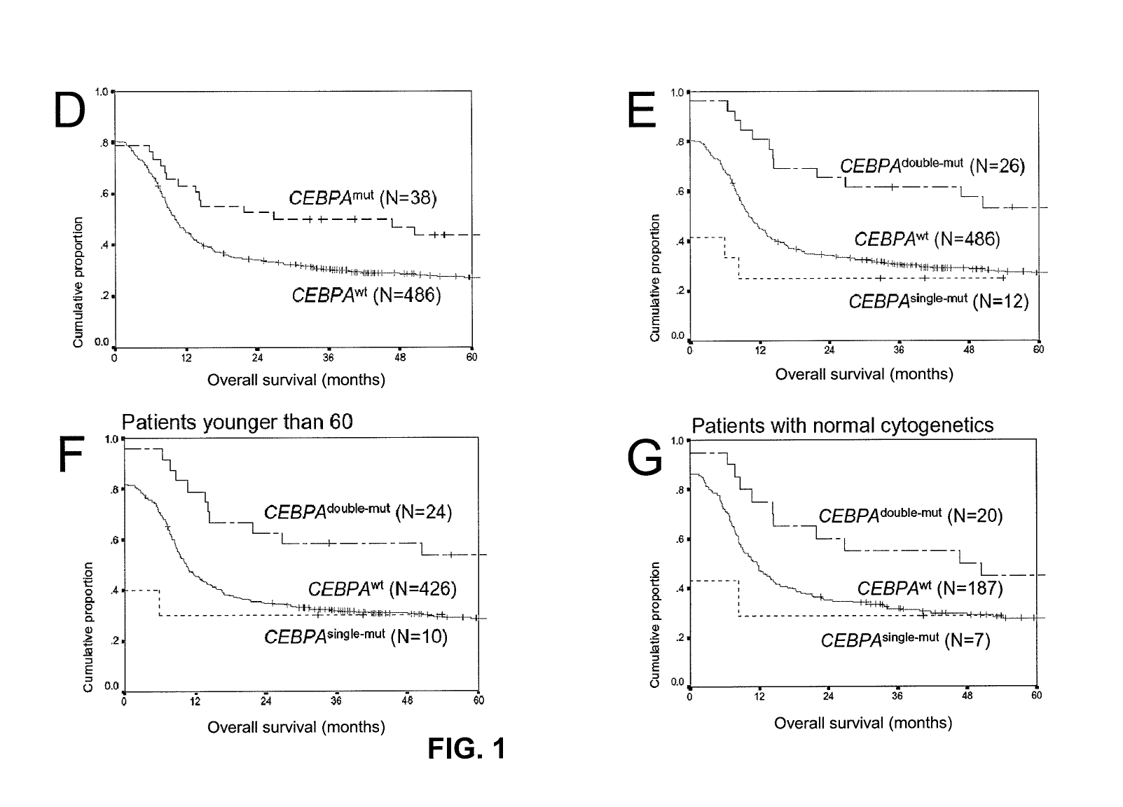

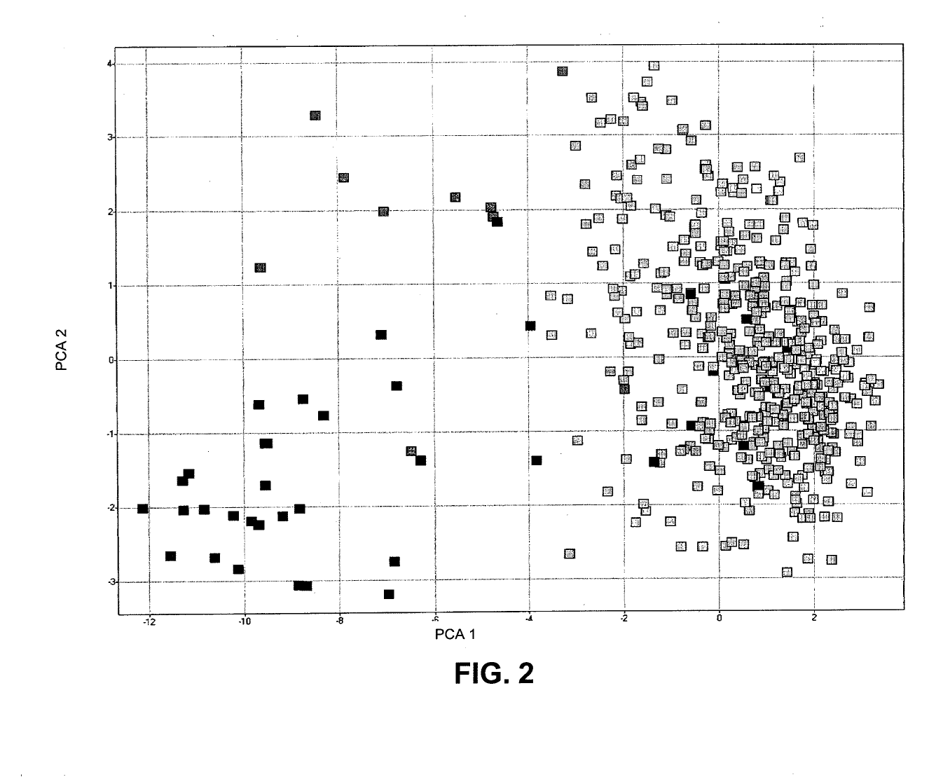

Complementary DNA (cDNA) was generated from 1 μg of mRNA using SuperScript reverse transcriptase (Invitrogen). The CEBPA coding region was divided into three overlapping amplicons (FIG. 1, Panel A). Primers for the three fragments (A, B and C) are shown in Table 14. PCR amplification for all three fragments was carried out using 2 μl of cDNA in mixes containing 0.5 mM dNTPs, 10% DMSO, 2 mM MgCl₂, 0.4 μM of forward and reverse primer, 1×PCR buffer and 2.5 units of Taq polymerase (Invitrogen), in a total volume of 50 microliters. Thermal cycling conditions for the three reactions were equal, i.e., denaturation at 94° C. for 5 minutes, followed by 35 cycles of 94° C. for 1 minute, 56° C. for 1 minute and 72° C. for 1 minute, and a final 5-minute elongation step of 72° C. After PCR amplification, 10 μl of PCR product was mixed with 10 μl of corresponding PCR product obtained from NB4 cell line cDNA. Heteroduplexes were allowed to form in an Applied Biosystems GeneAmp PCR System 9700 (two cycles of 95° C. for 3 minutes, cooled to 20° C. with a ramp of 5%, and maintained at 20° C. for 5 minutes). The samples were then subjected to denaturing high-performance liquid chromatography (DHPLC) analysis on a Transgenomics WAVE device, using temperatures of 65.4° C., 66.4° C. and 65.5° C., respectively. Data were analyzed using Transgenomics software, and aberrant peaks were independently scored by two investigators. Samples with aberrant peaks were subjected to direct nucleotide sequencing on an Applied Biosystems 3100 using the forward and reverse primers. In case a mutation was found, a second analysis on new input material was performed to rule out PCR-induced artifacts.

In AML cases for which dHPLC had revealed one single heterozygous mutation, the CEBPA coding region was fully sequenced to exclude the possibility that a second mutation had gone unnoticed. In three cases with an N-terminal mutation (#4336, #5362 and #5364), this extra analysis revealed an additional bZIP mutation. Two of these three were single nucleotide variations that were predicted to lead to substitutions of conserved amino acids in the basic region.⁴

Cases that appeared negative by dHPLC were additionally screened as follows. The CEBPA N-terminal part was nucleotide sequenced using previously described primers 2 and 10.⁵ Insertions or deletions in the basic leucine zipper domain were detected using a previously described ethidium bromide agarose gel electrophoresis approach and subsequent nucleotide sequencing (primers 4 and 8) in cases with apparent abnormalities.⁵

TABLE 14
Primer sequences

| | | | SEQ |
|---|---|---|---|
| Primer | Start (relative | | ID |
| name | to XM_009180.3) | Sequence (5′ to 3′) | NO: |
| A fw | 142 | CGCCATGCCGGGAGAACTCT | 157 |
| A rev | 400 | CTTCTCCTGCTGCCGGCTGT | 158 |
| B fw | 385 | GCCGCCTTCAACGACGAGTT | 159 |
| B rev | 643 | CTTGGCTTCATCCTCCTCGC | 160 |
| C fw | 634 | CGGCCGCTGGTGATCAAG | 161 |
| C rev | 1235 | CCCAGGGCGGTCCCACAGC | 162 |

### Statistical Analysis

Statistical analyses were performed in Statistical Package for the Social Sciences (SPSS) software, version 16.0. All patients received induction therapy and were included in the survival analysis. Actuarial probabilities of overall survival (OS, with death due to any cause) and event-free survival (EFS, with failure in case of no complete remission at day 1 [CR1] or relapse or death) were estimated by the method of Kaplan and Meier, and significance was assessed with the log rank test. Cox's proportional hazards models were fitted for multivariable analysis. Cytogenetic risk groups (favorable, intermediate, or poor) were defined as described.¹ Briefly, patients with inv(16)/t(16;16), t(8;21), and t(15;17) abnormalities, irrespective of the presence of additional cytogenetic aberrations, were considered as being in the favorable-risk category. These included a small number of cases in which the abnormality had been identified by RQ-PCR, despite noinial cytogenetics. The poor-risk category was defined by the presence of −5/del(5q), −7del(7q), t(6;9), t(9;22), 3q26 abnormality, or complex karyotype (more than three abnormalities) in the absence of good risk cytogenetic characteristics. All other patients were classified as intermediate risk. All tests were two-tailed, and a P value of less than 0.05 was considered statistically significant.

To investigate whether CEBPA mutations related to gene expression, we examined GEP data of 524 AML cases (including 26 CEBPA^{double-mut and} 12 CEBPA^{single-mut} cases). Clinical and molecular characteristics of the AML cases are depicted in Tables 3 and 4. Using a supervised approach, Prediction Analysis for Microarrays (PAM),¹⁷ we derived a 19-probe set signature predictive for CEBPA mutations (FIG. 1, Panel C). This classifier showed a high specificity (99%), but a limited sensitivity (67%) in cross-validation. Strikingly, misclassification was almost entirely due to CEBPA^{single-mut} cases, whereas CEBPA AMLs were predicted with an accuracy that was near perfect (FIG. 1, Panel C). In line with this, we were able to derive a specific CEBPA^{double-mut} classifier, consisting of 21 probe sets, with a cross-validated sensitivity of 100% (Table 5). In further support, unsupervised analysis of GEP data from the selected mutant subset confirmed an underlying variability that correlated with mutation status.

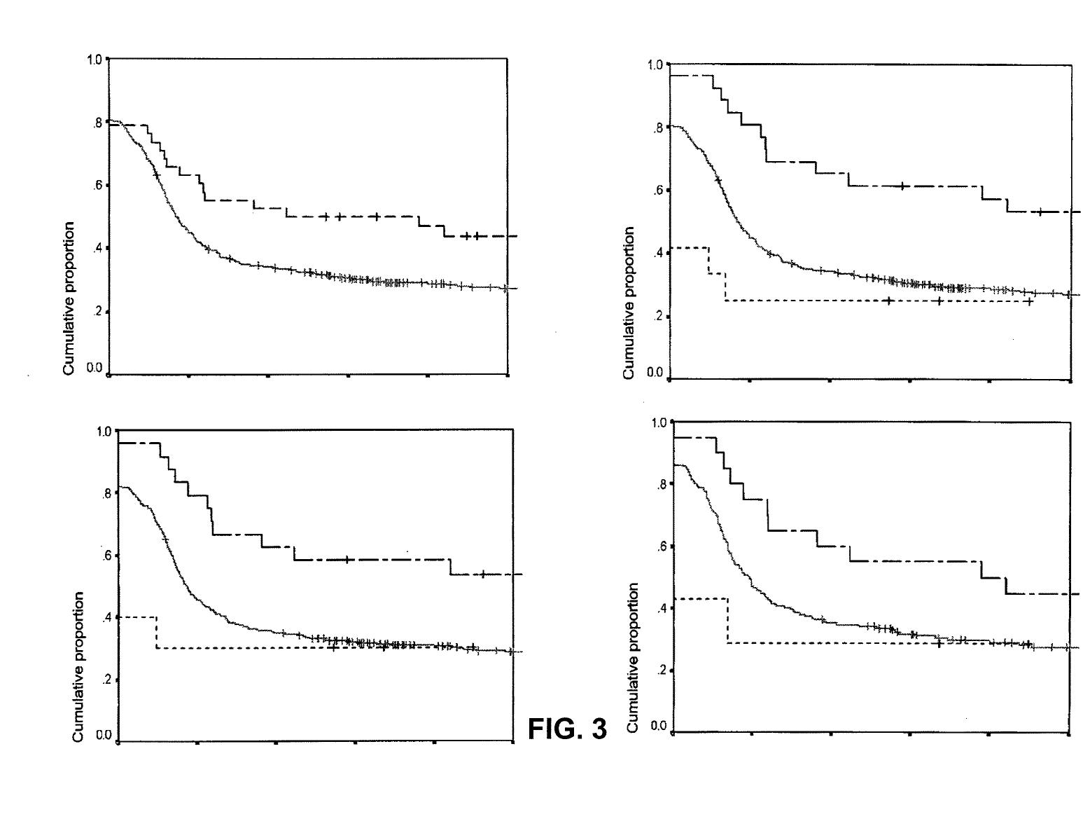

We next assessed whether our observations could be related to differences in clinical outcome. In line with previous data, overall survival (OS) and event-free survival (EFS) were better for CEBPA^{mut} cases compared to cases with wild-type CEBPA (CEBPA^{wt}) (FIG. 1, Panel D, and FIG. 3). Separate analyses for the two mutation subgroups, however, revealed a favorable outcome for CEBPA^{double-mut} cases but failed to find the same for the CEBPA^{single-mut} cases (FIG. 1, Panels E and G). In fact, CEBPA^{single-mut} AMLs showed a significantly worse _{outcome than CEBPA}^{double-mut} cases. These findings were retained in multivariable analyses (Table 1). When only patients younger than 60 were taken into account, similar results were found (FIG. 1, Panel F, Table 1). Likewise, in the selected subset of AML with normal cytogenetics, significant differences in OS and EFS were observed between CEBPA^{double-mut} and CEBPA^{single-mut} AMLs (FIG. 1, Panel G, Table 1).

Based on previous analyses in a subset of our cases³ and based on literature, it is likely that in the majority of CEBPA ^{double-mut} cases studied, both alleles were affected. A liable hypothesis is, therefore, that absence of wild-type CEBPA mRNA is directly involved in the CEBPA^{double-mut} gene expression profile. It is possible that analysis of larger patient series will lead to further refinement of the subclassification suggested here. Our data for instance indicated a tendency of CEBPA^{single-mut} cases with a mutation in the bZIP region, directly involved in DNA binding, to be potentially less distinct from the double mutants (cases #7185, #7324 and #2237; (FIG. 1, Panel C).

Studies to date have associated CEBPA mutations with outcome,^{3,4,9,18} but have not applied subdivisions into single and double mutants. It is unclear why AMLs with CEBPA^{double-mut} would have a better outcome than those with single mutations. One explanation could be that single mutations are not sufficient for leukemogenesis and require additional mutations. In possible support of this hypothesis, we found significantly more FLT3-ITD, FLT3-TKD and NPM1 mutations in CEBPA^{single-mut} compared to CEBPA^{double-mut} cases (Table 4). Currently unknown abnormalities may associate with CEBPA^{single-mut} AML as well and predispose to relatively inferior outcome. It seems evident, however, that these findings and their clinical significance warrant further investigation and confirmation in independent series of AML.

In summary, the data presented here indicate that CEBPA mutant AML should not be considered a single biologic and clinical group but at least be distinguished according the presence of CEBPA^{double-mut} and CEBPA^{single-mut}. We suggest that screening using dHPLC, followed by nucleotide sequencing, should rapidly identify mutant cases. Second, gene expression based classification, for instance, using the classifiers described here, should allow accurate identification of CEBPA^{double-mut} AML cases.

### U133Plus2 GeneChip Gene Expression Profiling Analysis

Raw microarray data were processed using Affymetrix Microarray Suite 5 (MASS) to target intensity values of 100. Intensity values lower than 30 were set at 30, and subsequently all data were log2 transformed.

Gene expression classifiers for CEBPA^{mut} and CEBPA^{double-mut} were derived using Prediction Analysis for Microarrays (PAM)⁶ version 1.28 in R version 2.1.0. The method of the nearest shrunken centroids identifies a subgroup of genes that best characterizes a predefined class. In accordance with good practice guidelines,⁷′⁸ all available data were used for classifier construction, and estimated predictive performances were based on cross-validation as follows. PAM was first used to train a classifier based on the entire data set of 524 AML cases. Next, selection of a shrinkage factor (in order to only use the most informative genes) as well as estimation of classifier performance were carried out using ten-fold cross-validation, involving a random split of the data into ten folds, which was balanced with respect to mutation status. Each fold was once used as an independent validation set for a classifier that has been trained on the remaining nine folds. The minimum number of misclassified cases was subsequently determined, and the corresponding shrinkage threshold was recorded. Furthermore, sensitivity and specificity were calculated. This entire procedure of ten-fold random cross-validation was repeated 100 times. Reported final classifiers represent the probe sets that remained after shrinkage using the median threshold over the 100 cross-validations. Reported final sensitivities and specificities represent the averages over the 100-cross-validations. Criterion for the CEBPA^{mut} classifier was minimum total misclassification rate (i.e., minimum false positives+false negatives). Criterion for the reported CEBP ^{double-mut} classifier was minimum misclassification of double mutant specimens (i.e., minimum false negatives).

Principal component analysis was performed using Spotfire Decision Site (Spotfire, Inc., Somerville, Mass.). Before the analysis, data for all probe sets were mean-centered.

### CEPBA^{double-mut} Analysis with the AMLProfiler

In addition to the detection of CEBPA^{double-mut} among results obtained with the U133Plus2 GeneChip platform, we have also hybridized 505 of the above 598 AML cases on the AMLProfiler to optimize the procedure for this platform. Also, we have improved the performance of the procedure through addition of a gene expression level pre-filtering. After normalization, scaling, imputation, intensity mean centering, and log2 transformation, the first criterion is that all CEBPA^{double-mut} have a gene expression above a certain threshold for the CEBPA gene itself. Next, a LDA classifier decides whether a sample is CEBPA^{double-mut} or not. This has shown to be the most efficient method so far. Details of the sequences used are provided in the various tables.

These results show that the selection of classifier genes is independent from the platform used for determining the expression levels of the genes identified herein. Hence, the teaching of this patent application may be extended to any platform. However, the U133Plus2 Genechip platform and the AML Profiler platform remain preferred.

### AMLProfiler GeneChip Gene Expression Profiling Analysis

Raw microarray data were processed using Affymetrix Microarray Suite 5 (MASS) to target intensity values of 1500. Intensity values lower than 30 were set at 30, and subsequently all data were log2 transformed. The data was mean-centered per probe. All computational analyses were performed using R (www.r-project.org, version 2.9.2) or Matlab (www.mathworks.com, version R2009a).

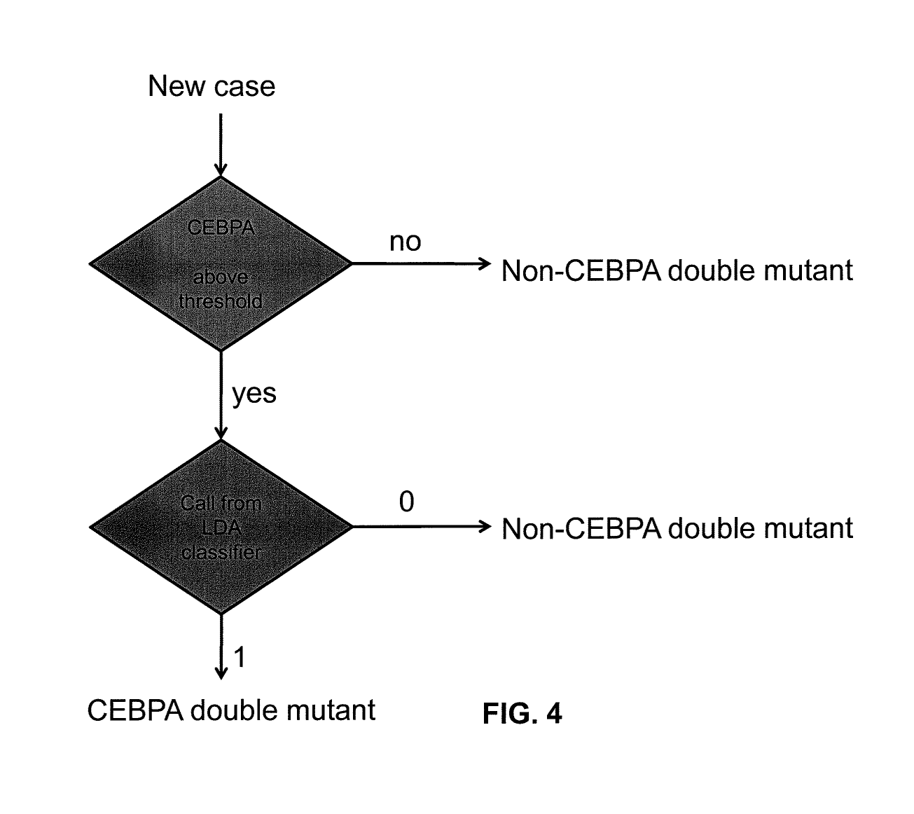

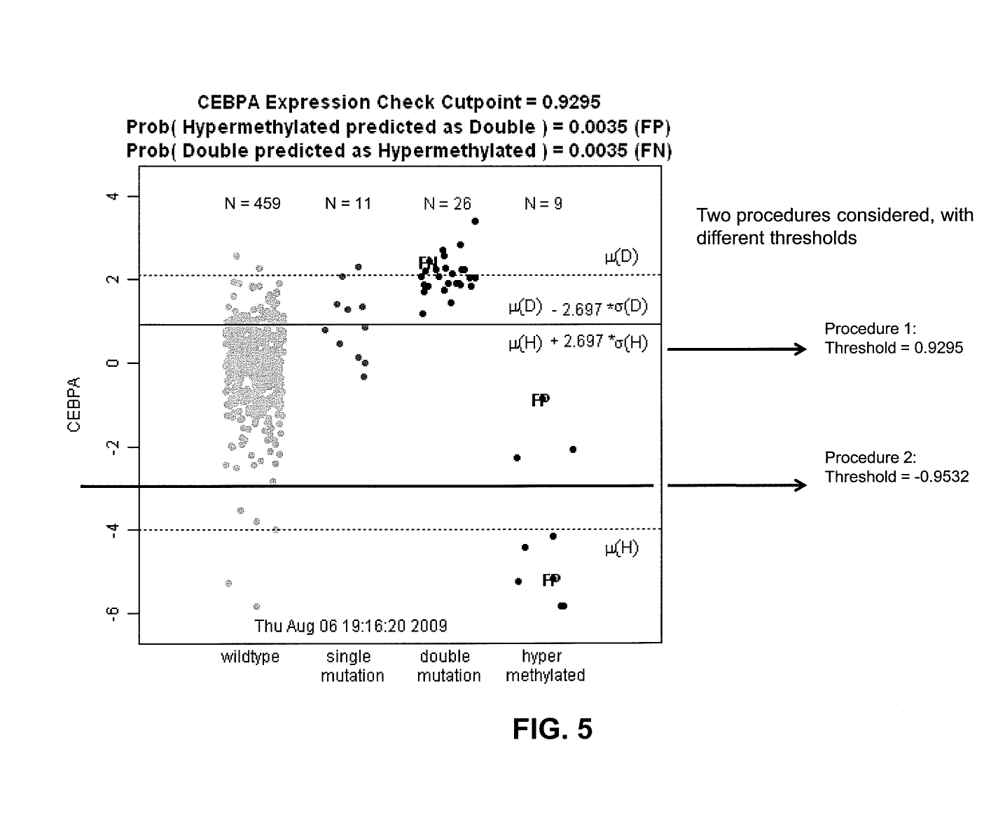

The procedure to detect the CEBPA^{double-mut} status consists of two serial steps (see FIG. 4). In the first step, samples with a CEBPA expression below a threshold are all predicted as non-double mutant. In the second step, a classifier is trained to predict CEBPA^{double-mut} vs non-CEBPA^{double-mut} (linear classifier, LDA, Dabney et al., Bioinformatics, 2005). Currently, two procedures are preferred, differing in the chosen threshold in the first step, and consequentially have a slightly different classifier in the second step (see FIG. 5).

Based on a double-loop cross-validation protocol (DLCV, Wessels et al., Bioinformatics, 2005), we determined an optimal set of genes for the classifier. This DLCV was run with 100 repeats of each of 26 folds in the outer loop, and ten-fold cross-validation in the inner loop. Probes were ranked univariately (t-test, equal variance), and learning curves were constructed for up to 50 probes. The classifier was optimized such that the average false positive ratio/false negative ratio was minimal. Reported final signatures were derived using all samples, using the number of features estimated in the DLCV.

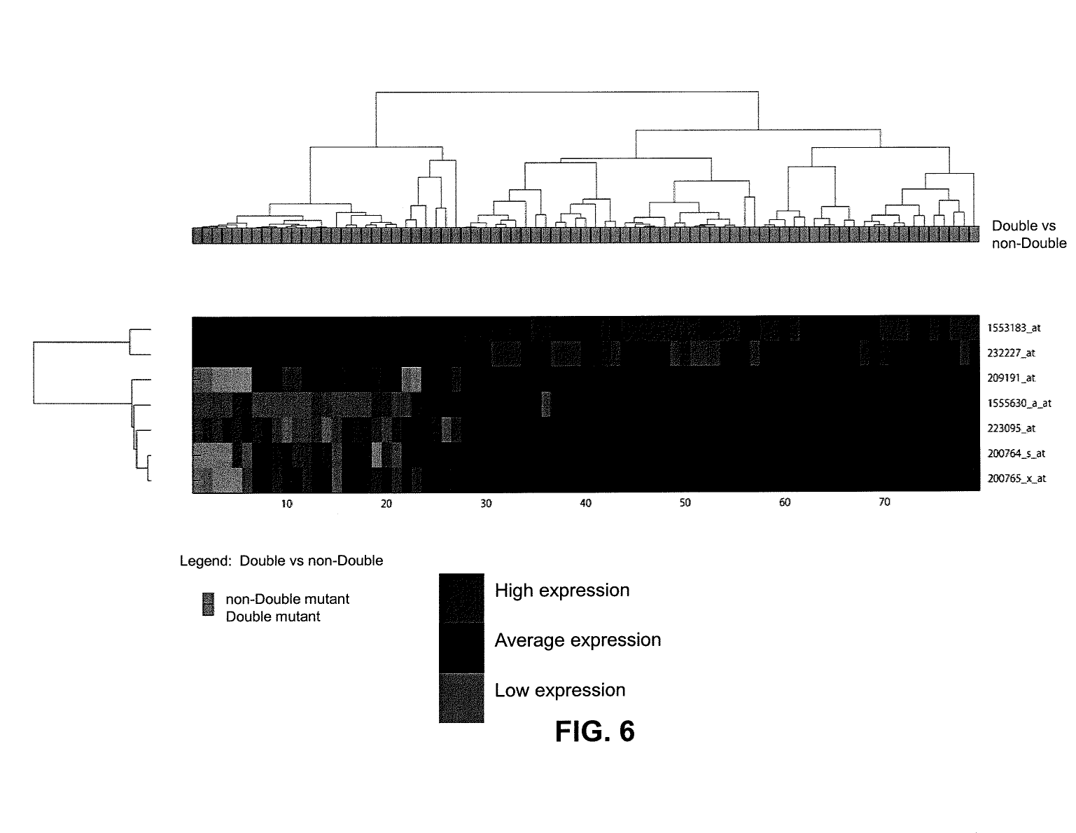

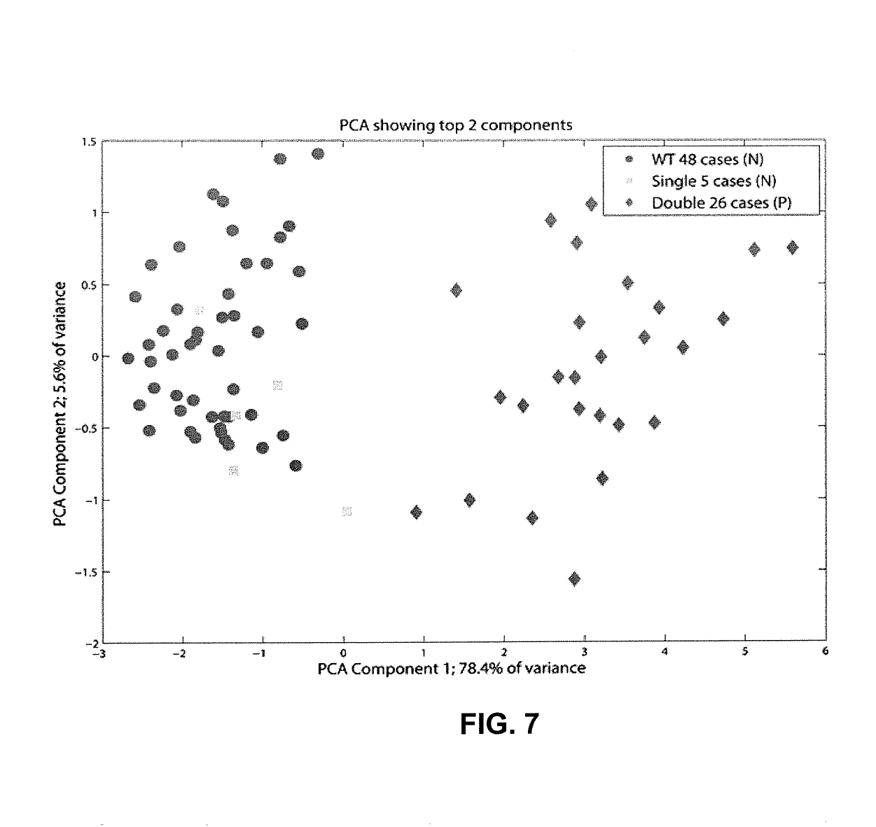

For procedure 1, we chose a threshold value of t=0.9295 such that all hypermethylated samples are well below the threshold (see FIG. 6). Subsequently, a classifier set of seven genes was used in a method according to the invention. Both samples and genes were hierarchically clustered (see FIG. 6), and a PCA plot was constructed (see FIG. 7). In addition, Table 9 lists the seven probes with extensive annotation.

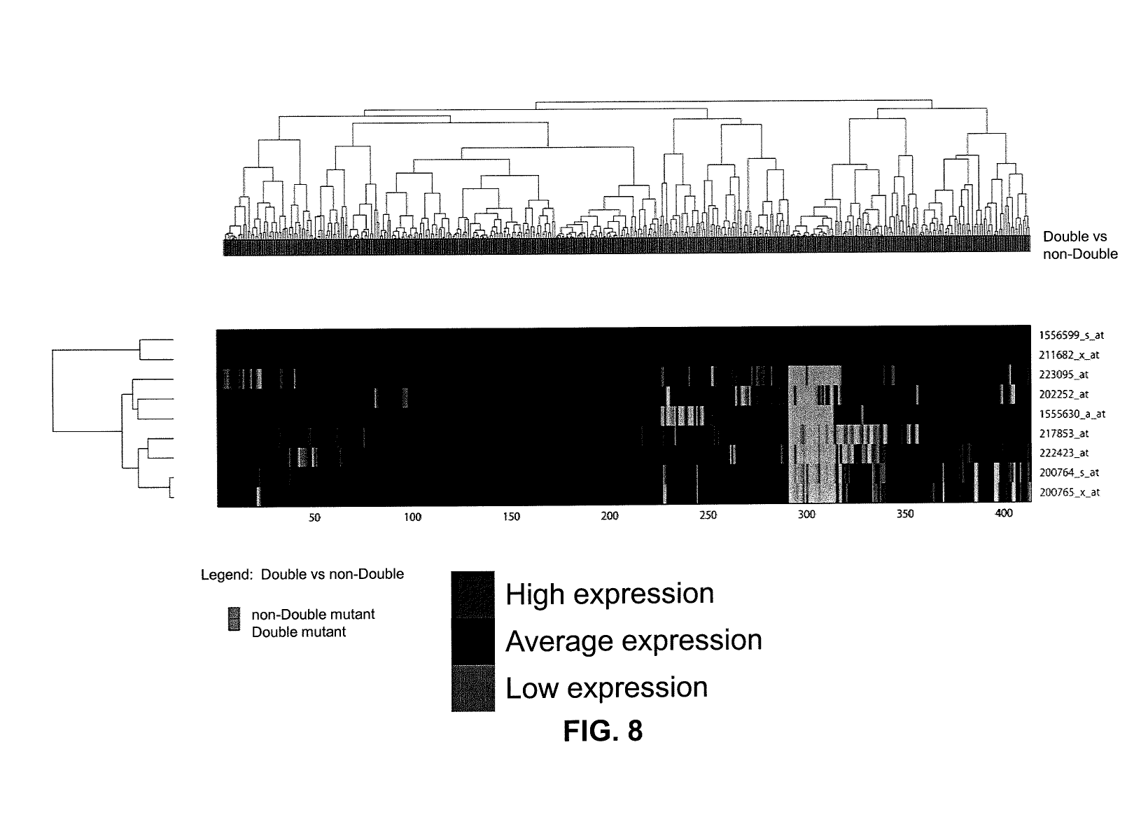

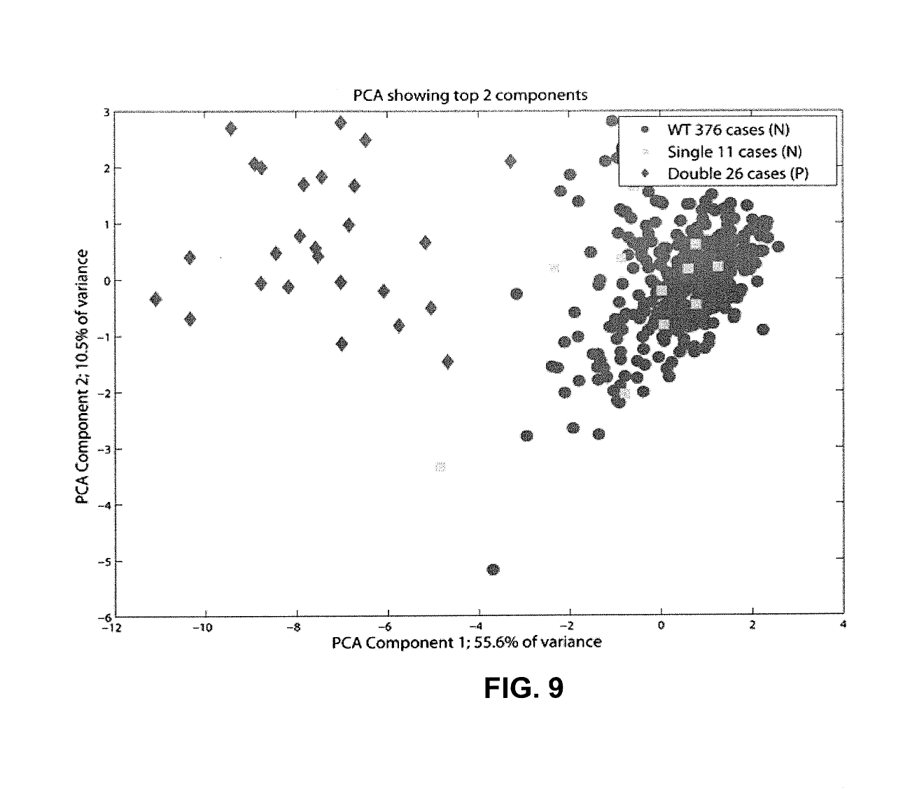

For procedure 2, we chose a threshold t=−0.9532. We determined an optimal set of nine genes for the classifier. Both samples and genes were hierarchically clustered (see FIG. 8), and a PCA plot was constructed (see FIG. 9). In addition, Table 10 lists the nine probes with extensive annotation.

### REFERENCES FOR THE EXAMPLES SECTION

1. Verhaak R. G., C. S. Goudswaard, W. van Putten, et al. Mutations in nucleophosmin

(NPM1) in acute myeloid leukemia (AML): association with other gene abnomialities and previously established gene expression signatures and their favorable prognostic significance. Blood 2005, 106:3747-3754.

2. Valk P. J., D. T. Bowen, M. E. Frew, A. C. Goodeve, B. Lowenberg, and J. T. Reilly. Second hit mutations in the RTK/RAS signaling pathway in acute myeloid leukemia with inv(16). Haematologica 2004, 89:106.

3. Care R. S., P. J. Valk, A. C. Goodeve, et al. Incidence and prognosis of c-KIT and FLT3 mutations in core binding factor (CBF) acute myeloid leukaemias. Br. J. Haematol. 2003, 121:775-777.

4. Vinson C. R., P. B. Sigler, and S. L. McKnight. Scissors-grip model for DNA recognition by a family of leucine zipper proteins. Science 1989, 246:911-916.

5. Barjesteh van Waalwijk van Doorn-Khosrovani S., C. Erpelinck, J. Meijer, et al. Biallelic mutations in the CEBPA gene and low CEBPA expression levels as prognostic markers in intermediate-risk AML. Hematol. J. 2003, 4:31-40.

6. Tibshirani R., T. Hastie, B. Narasimhan, and G. Chu. Diagnosis of multiple cancer types by shrunken centroids of gene expression. Proc. Natl. Acad. Sci. U.S A. 2002, 99:6567-6572.

7. Simon R. Roadmap for developing and validating therapeutically relevant genomic classifiers. J. Clin. Oncol. 2005, 23:7332-7341.

8. Dupuy A., and R. M. Simon. Critical review of published microarray studies for cancer outcome and guidelines on statistical analysis and reporting. J. Natl. Cancer Inst. 2007, 99:147-157.

### REFERENCES FOR THE BODY OF THE DESCRIPTION

1. Zhang D. E., P. Zhang, N. D. Wang, C. J. Hetherington, G. J. Darlington, and D. G. Tenen. Absence of granulocyte colony-stimulating factor signaling and neutrophil development in CCAAT enhancer binding protein alpha-deficient mice. Proc. Natl. Acad. Sci. U.S.A. 1997, 94:569-574.

2. Zhang P., J. Iwasaki-Arai, H. Iwasaki, et al. Enhancement of hematopoietic stem cell repopulating capacity and self-renewal in the absence of the transcription factor C/EBP alpha. Immunity 2004, 21:853-863.

3. Barjesteh van Waalwijk van Doom-Khosrovani S., C. Erpelinck, J. Meijer, et al. Biallelic mutations in the CEBPA gene and low CEBPA expression levels as prognostic markers in intermediate-risk AML. Hematol. J. 2003, 4:31-40.

4. Frohling S., R. F. Schlenk, I. Stoize, et al. CEBPA mutations in younger adults with acute myeloid leukemia and normal cytogenetics: prognostic relevance and analysis of cooperating mutations. J. Clin. Oncol. 2004, 22:624-633.

5. Gombart A. F., W. K. Hofmann, S. Kawano, et al. Mutations in the gene encoding the transcription factor CCAAT/enhancer binding protein alpha in myelodysplastic syndromes and acute myeloid leukemias. Blood 2002, 99:1332-1340.

6. Pabst T., B. U. Mueller, P. Zhang, et al. Dominant-negative mutations of CEBPA, encoding CCAAT/enhancer binding protein-alpha (C/EBPalpha), in acute myeloid leukemia. Nat. Genet. 2001, 27:263-270.

7. Nerlov C. C/EBPalpha mutations in acute myeloid leukaemias. Nat. Rev. Cancer 2004, 4:394-400.

8. Leroy H., C. Roumier, P. Huyghe, V. Biggio, P. Fenaux, and C. Preudhomme. CEBPA point mutations in hematological malignancies. Leukemia 2005, 19:329-334.

9. Preudhomme C., C. Sagot, N. Boissel, et al. Favorable prognostic significance of CEBPA mutations in patients with de novo acute myeloid leukemia: a study from the Acute Leukemia French Association (ALFA). Blood 2002, 100:2717-2723.

10. Schlenk R. F., K. Dohner, J. Krauter, et al. Mutations and treatment outcome in cytogenetically normal acute myeloid leukemia. N. Engl. J. Med. 2008, 358:1909-1918.

11. Lin L. I., C. Y. Chen, D. T. Lin, et al. Characterization of CEBPA mutations in acute myeloid leukemia: most patients with CEBPA mutations have biallelic mutations and show a distinct immunophenotype of the leukemic cells. Clin. Cancer Res. 2005, 11:1372-1379.

12. Valk P. J., R. G. Verhaak, M. A. Beijen, et al. Prognostically useful gene-expression profiles in acute myeloid leukemia. N. Engl. J. Med. 2004, 350:1617-1628.

13. Verhaak R. G., C. S. Goudswaard, W. van Putten, et al. Mutations in nucleophosmin (NPM1) in acute myeloid leukemia (AML): association with other gene abnormalities and previously established gene expression signatures and their favorable prognostic significance. Blood 2005, 106:3747-3754.

14. Wouters B. J., I. Louwers, P. J. Valk, B. Lowenberg, and R. Delwel. A recurrent in-frame insertion in a CEBPA transactivation domain is a polymorphism rather than a mutation that does not affect gene expression profiling-based clustering of AML. Blood 2007, 109:389-390.

15. Resende C., G. Regalo, C. Duraes, F. Carneiro, and J. C. Machado. Genetic changes of CEBPA in cancer: mutations or polymorphisms? J. Clin. Oncol. 2007, 25:2493-2494; author reply 2494-2495.

16. Biggio V., A. Renneville, O. Nibourel, et al. Recurrent in-frame insertion in C/EBPalpha

TAD2 region is a polymorphism without prognostic value in AML. Leukemia 2008, 22:655-657.

17. Tibshirani R., T. Hastie, B. Narasimhan, and G. Chu. Diagnosis of multiple cancer types by shrunken centroids of gene expression. Proc. Natl. Acad. Sci. U.S.A. 2002, 99:6567-6572.

18. Bienz M., M. Ludwig, E. O. Leibundgut, et al. Risk assessment in patients with acute myeloid leukemia and a normal karyotype. Clin. Cancer Res. 2005, 11:1416-1424.

19. Wouters B. J., M. A. Jorda, K. Keeshan, et al. Distinct gene expression profiles of acute myeloid/T-lymphoid leukemia with silenced CEBPA and mutations in NOTCH1. Blood 2007, 110:3706-3714.

1. A method for determining whether a subject carries a biallelic CCAAT/enhancer binding protein alpha (“CEBPA”) mutation, the method comprising: determining, in a sample obtained from the subject, the expression levels of at least 2 genes selected from the group consisting of NDFIP1, RAB34, UGT2B28, MARVELD1, RAB13, UMODL1, TNS3, LOC136306, HSPB1///MEIS3, LRRC28, CTNNA1, SFXN3, GLULD1, DLC1, SUCLG2, PODS, HSPC324, TUBB6, ARPP-21, and CEBPA.

2. The method according to claim 1, wherein the at least 2 genes are selected from the group consisting of NDFIP1, RAB34, UGT2B28, MARVELD1, RAB13, UMODL1, TNS3, LOC136306, HSPB1///MEIS3, LRRC28, CTNNA1, SFXN3, GLULD1, DLC1, SUCLG2, PGDS, HSPC324, TUBB6, ARPP-21, SHD, and CEBPA.

3. The method according to claim 1, wherein the at least 2 genes are selected from the group consisting of NDFIP1, RAB34, UGT2B28/B10, MARVELD1, RAB13, UMODL1, TNS3, CTNNA1, HSPC324, TUBB6, ARPP-21, and CEBPA.

4. The method according to claim 1, wherein the at least 2 genes are selected from the group consisting of RAB34, MARVELD1, UMODL1, CTNNA1, HSPC324, TUBB6, and CEBPA.

5. The method according to claim 1, wherein the at least 2 genes are selected from the group consisting of NDFIP 1, RAB34, UGT2B28/B10, MARVELD1, RAB13, TNS3, CTNNA1, ARPP-21, and CEBPA.

6. The method according to claim 4, further comprising determining the expression level level of each of RAB34, MARVELD1, UMODL1, CTNNA1, HSPC324, TUBB6, and CEBPA.

7. The method according to claim 5, further comprising determining the expression level of each of NDFIP1, RAB34, UGT2B28/B10, MARVELD1, RAB13, TNS3, CTNNA1, ARPP-21, and CEBPA.

8. The method according to claim 4, further comprising determining the expression level of CEBPA and comparing the expression level of CEBPA to a predetermined value.

9. The method according to claim 5, further comprising determining the expression level of CEBPA and comparing the expression level of CEBPA to a predetermined value.

10. The method according to claim 1 wherein the sample is taken from the group consisting of a tissue sample, a blood sample, a urine sample or a sputum sample


SEQUENCE LISTING (162 sequences)

SEQ ID NO: 1 - DNA, 25 bp, Homo sapiens
aagctaggtcgtgggtcagctctga

SEQ ID NO: 2 - DNA, 25 bp, Homo sapiens
agctctgaggatgtatacccctggt

SEQ ID NO: 3 - DNA, 25 bp, Homo sapiens
gagggagacctagagatctggctgt

SEQ ID NO: 4 - DNA, 25 bp, Homo sapiens
agccttgtttgtactgtatgccttc

SEQ ID NO: 5 - DNA, 25 bp, Homo sapiens
atgccttcagcattgcctaggaaca

SEQ ID NO: 6 - DNA, 25 bp, Homo sapiens
gaacacgaagcacgatcagtccatc

SEQ ID NO: 7 - DNA, 25 bp, Homo sapiens
tcaacacttgtatctggcctctgtg

SEQ ID NO: 8 - DNA, 25 bp, Homo sapiens
tgtgaatgtaccgtctctgctaaac

SEQ ID NO: 9 - DNA, 25 bp, Homo sapiens
tgtttggttttctcggatacttgcc

SEQ ID NO: 10 - DNA, 25 bp, Homo sapiens
gccaaaatgagactctccgtcggca

SEQ ID NO: 11 - DNA, 25 bp, Homo sapiens
ccctggctcctggtcgggaagggag

SEQ ID NO: 12 - DNA, 25 bp, Homo sapiens
tgcctgtagcattccagagctcact

SEQ ID NO: 13 - DNA, 25 bp, Homo sapiens
ctcactctgtctgccaggtatgaga

SEQ ID NO: 14 - DNA, 25 bp, Homo sapiens
agaagaacacgtaagaccgccacca

SEQ ID NO: 15 - DNA, 25 bp, Homo sapiens
ctgcccccagaacttttggatactg

SEQ ID NO: 16 - DNA, 25 bp, Homo sapiens
ggaggcagttgcataggtctccctc

SEQ ID NO: 17 - DNA, 25 bp, Homo sapiens
gcccaagactactctgggcagctcc

SEQ ID NO: 18 - DNA, 25 bp, Homo sapiens
ggaaaggatttctacagtgttctat

SEQ ID NO: 19 - DNA, 25 bp, Homo sapiens
agagagagtgggtttgggaagagtg

SEQ ID NO: 20 - DNA, 25 bp, Homo sapiens
gttggggagaggggaccgatgtgcc

SEQ ID NO: 21 - DNA, 25 bp, Homo sapiens
gaccgatgtgcctcattgtttagtg

SEQ ID NO: 22 - DNA, 25 bp, Homo sapiens
ttctggataaagtttggttgtttgc

SEQ ID NO: 23 - DNA, 25 bp, Homo sapiens
aatgcaggagcctacttctagctgt

SEQ ID NO: 24 - DNA, 25 bp, Homo sapiens
gaccaagccgagatgccattctctt

SEQ ID NO: 25 - DNA, 25 bp, Homo sapiens
ttctcttagtgatggcggcgttagg

SEQ ID NO: 26 - DNA, 25 bp, Homo sapiens
gttgagagggtgcagtccagacagc

SEQ ID NO: 27 - DNA, 25 bp, Homo sapiens
gtccagacagcttgactgcttttaa

SEQ ID NO: 28 - DNA, 25 bp, Homo sapiens
gacctgtggtaagcaacctggcatc

SEQ ID NO: 29 - DNA, 25 bp, Homo sapiens
ttactaatgtacgctgctgcaggac

SEQ ID NO: 30 - DNA, 25 bp, Homo sapiens
gctacagtgtctcgatgccataatc

SEQ ID NO: 31 - DNA, 25 bp, Homo sapiens
tcagaacacactttttttcctcttt

SEQ ID NO: 32 - DNA, 25 bp, Homo sapiens
tttctcccagcttcaaatgcacaat

SEQ ID NO: 33 - DNA, 25 bp, Homo sapiens
aattcatcattgggctcacttctaa

SEQ ID NO: 34 - DNA, 25 bp, Homo sapiens
aggtgcagaatctcggcggggagct

SEQ ID NO: 35 - DNA, 25 bp, Homo sapiens
gaatctcggcggggagcttgttgtc

SEQ ID NO: 36 - DNA, 25 bp, Homo sapiens
gatccaggcagccaagaacttgatg

SEQ ID NO: 37 - DNA, 25 bp, Homo sapiens
aacaggatgagacacagaccaagat

SEQ ID NO: 38 - DNA, 25 bp, Homo sapiens
tggacagcatctaagtctgcccagg

SEQ ID NO: 39 - DNA, 25 bp, Homo sapiens
ggctcctgaatatcagtcactgttc

SEQ ID NO: 40 - DNA, 25 bp, Homo sapiens
cagtcactgttcgtcactcaaatga

SEQ ID NO: 41 - DNA, 25 bp, Homo sapiens
tacaacactgatactagattccaca

SEQ ID NO: 42 - DNA, 25 bp, Homo sapiens
ggaaatgggcagactgaaccagtcc

SEQ ID NO: 43 - DNA, 25 bp, Homo sapiens
gaaccagtccaggtggtgaattttc

SEQ ID NO: 44 - DNA, 25 bp, Homo sapiens
aatgcaggagcctacttctagctgt

SEQ ID NO: 45 - DNA, 25 bp, Homo sapiens
aatgcattgcatcaacctactatag

SEQ ID NO: 46 - DNA, 25 bp, Homo sapiens
ggagctcaagccatcatcattgtct

SEQ ID NO: 47 - DNA, 25 bp, Homo sapiens
gtggcatctctggaacataccaagc

SEQ ID NO: 48 - DNA, 25 bp, Homo sapiens
ggagaatgacccttccagtgtgctt

SEQ ID NO: 49 - DNA, 25 bp, Homo sapiens
tgcttctcttccttgtaggttccaa

SEQ ID NO: 50 - DNA, 25 bp, Homo sapiens
gttccaagaaggatctgagtacccc

SEQ ID NO: 51 - DNA, 25 bp, Homo sapiens
ccctgctcagtatgcgctgatggag

SEQ ID NO: 52 - DNA, 25 bp, Homo sapiens
ccctggacatttgcactgactttat

SEQ ID NO: 53 - DNA, 25 bp, Homo sapiens
agctgggatcatgctagtcacttcc

SEQ ID NO: 54 - DNA, 25 bp, Homo sapiens
ggagagtgtctctgttcttttgctc

SEQ ID NO: 55 - DNA, 25 bp, Homo sapiens
ggatgcttaatgattccagcctctc

SEQ ID NO: 56 - DNA, 25 bp, Homo sapiens
gagggagaactttcctgtggacgtc

SEQ ID NO: 57 - DNA, 25 bp, Homo sapiens
tcaaccgagggggaggtcacttccg

SEQ ID NO: 58 - DNA, 25 bp, Homo sapiens
tcacttccgaatccacagatggcgt

SEQ ID NO: 59 - DNA, 25 bp, Homo sapiens
gatggcgtgtgagtgcatggcgagc

SEQ ID NO: 60 - DNA, 25 bp, Homo sapiens
ctccaggacacacttactgttccct

SEQ ID NO: 61 - DNA, 25 bp, Homo sapiens
ctgctctttgcagctgcctgcaagg

SEQ ID NO: 62 - DNA, 25 bp, Homo sapiens
tgcaaggggttcctgcgaagaccag

SEQ ID NO: 63 - DNA, 25 bp, Homo sapiens
agagcctgcggctgaacttgaactc

SEQ ID NO: 64 - DNA, 25 bp, Homo sapiens
gaactcgcagctacctgagtcagac

SEQ ID NO: 65 - DNA, 25 bp, Homo sapiens
tgagtcagacctgtgctttttcacc

SEQ ID NO: 66 - DNA, 25 bp, Homo sapiens
atgtcagagcgtttccctagcaatg

SEQ ID NO: 67 - DNA, 25 bp, Homo sapiens
cttttctcctgtgtaccaaggtatt

SEQ ID NO: 68 - DNA, 25 bp, Homo sapiens
tttatttacacgacagcgactcaaa

SEQ ID NO: 69 - DNA, 25 bp, Homo sapiens
gactcaaaaggcactcgattaatgt

SEQ ID NO: 70 - DNA, 25 bp, Homo sapiens
atgtgacaaccttttcaataagcag

SEQ ID NO: 71 - DNA, 25 bp, Homo sapiens
gtaggtacacatcactctttacatt

SEQ ID NO: 72 - DNA, 25 bp, Homo sapiens
agcattttcacagccgtttcttcat

SEQ ID NO: 73 - DNA, 25 bp, Homo sapiens
catataatccgaccacagtgggagg

SEQ ID NO: 74 - DNA, 25 bp, Homo sapiens
ggaggtgtgatttacccattacaca

SEQ ID NO: 75 - DNA, 25 bp, Homo sapiens
ccagaggagccgatgagttacttaa

SEQ ID NO: 76 - DNA, 25 bp, Homo sapiens
agaattttctccattacatcatcct

SEQ ID NO: 77 - DNA, 25 bp, Homo sapiens
gataatgctctgtacttcatttgaa

SEQ ID NO: 78 - DNA, 25 bp, Homo sapiens
gatagtcggaatagagccgccccaa

SEQ ID NO: 79 - DNA, 25 bp, Homo sapiens
aactcagatcctacaacacgcaagt

SEQ ID NO: 80 - DNA, 25 bp, Homo sapiens
caacacgcaagttccttcttgaacc

SEQ ID NO: 81 - DNA, 25 bp, Homo sapiens
ccctatggccctgaatggtgcactg

SEQ ID NO: 82 - DNA, 25 bp, Homo sapiens
cccctcacaaatgcagccaagtcat

SEQ ID NO: 83 - DNA, 25 bp, Homo sapiens
gagaattgcgggttctacccagtca

SEQ ID NO: 84 - DNA, 25 bp, Homo sapiens
ctacccagtcagaagatcacaccat

SEQ ID NO: 85 - DNA, 25 bp, Homo sapiens
gatcacaccatggagactttctact

SEQ ID NO: 86 - DNA, 25 bp, Homo sapiens
aacttcaccttccattaagtgttca

SEQ ID NO: 87 - DNA, 25 bp, Homo sapiens
gaagcctgaaattgtgccgtgttgc

SEQ ID NO: 88 - DNA, 25 bp, Homo sapiens
tgccgtgttgccttatatgaatatg

SEQ ID NO: 89 - DNA, 25 bp, Homo sapiens
gagtacagacctgctgaatgcactg

SEQ ID NO: 90 - DNA, 25 bp, Homo sapiens
tgatcaaccagtaaagcccctgcat

SEQ ID NO: 91 - DNA, 25 bp, Homo sapiens
cccctgcatcgagcagtcttctgga

SEQ ID NO: 92 - DNA, 25 bp, Homo sapiens
aaggagccaaacaccttcgagttgc

SEQ ID NO: 93 - DNA, 25 bp, Homo sapiens
ggttccagtaccactctttggatgt

SEQ ID NO: 94 - DNA, 25 bp, Homo sapiens
ggatgtgattgggtttctgctggcc

SEQ ID NO: 95 - DNA, 25 bp, Homo sapiens
ctgctggcctgtgtggcaactgtga

SEQ ID NO: 96 - DNA, 25 bp, Homo sapiens
ttgtctgttttgtttctggaagttt

SEQ ID NO: 97 - DNA, 25 bp, Homo sapiens
gggttgacatcagtttattccagca

SEQ ID NO: 98 - DNA, 25 bp, Homo sapiens
agatttctttcttcctgtgacaaaa

SEQ ID NO: 99 - DNA, 25 bp, Homo sapiens
tttttcagagatttaccacccaggt

SEQ ID NO: 100 - DNA, 25 bp, Homo sapiens
caggaaacggcaacaagcctcccag

SEQ ID NO: 101 - DNA, 25 bp, Homo sapiens
gcctcccagtactgacctgaaaact

SEQ ID NO: 102 - DNA, 25 bp, Homo sapiens
gaagaacaccaacaagtgctccctg

SEQ ID NO: 103 - DNA, 25 bp, Homo sapiens
caccccggaagctgaacctgaggga

SEQ ID NO: 104 - DNA, 25 bp, Homo sapiens
gagagatgcctcaggcttcagacct

SEQ ID NO: 105 - DNA, 25 bp, Homo sapiens
cttcagaccttacctgggttttcag

SEQ ID NO: 106 - DNA, 25 bp, Homo sapiens
agggtcctgcaaaaggctagctcgg

SEQ ID NO: 107 - DNA, 25 bp, Homo sapiens
gctagctcggcactacactagggaa

SEQ ID NO: 108 - DNA, 25 bp, Homo sapiens
actagggaatttgctcctgttctgt

SEQ ID NO: 109 - DNA, 25 bp, Homo sapiens
tcacttgtcatggtctttcttggta

SEQ ID NO: 110 - DNA, 25 bp, Homo sapiens
ggtattaaaggccaccatttgcaca

SEQ ID NO: 111 - DNA, 25 bp, Homo sapiens
tctgctgcatttctaatgtgaattg

SEQ ID NO: 112 - DNA, 25 bp, Homo sapiens
gagcttatgaatacactcttttcaa

SEQ ID NO: 113 - DNA, 25 bp, Homo sapiens
tcaagtgtatgtgggttttcaaggt

SEQ ID NO: 114 - DNA, 25 bp, Homo sapiens
gtttgatcctatatttcctttgtta

SEQ ID NO: 115 - DNA, 25 bp, Homo sapiens
ctgcttttcatcactttgacagaat

SEQ ID NO: 116 - DNA, 25 bp, Homo sapiens
gagtttgggtatatgctttgaggaa

SEQ ID NO: 117 - DNA, 25 bp, Homo sapiens
ggaatatgcttcctcattacaaaag

SEQ ID NO: 118 - DNA, 25 bp, Homo sapiens
gattactacacttgcttatcaatag

SEQ ID NO: 119 - DNA, 25 bp, Homo sapiens
aagatacgtggattgctatgcattc

SEQ ID NO: 120 - DNA, 25 bp, Homo sapiens
atttatcctctccaggcatgattta

SEQ ID NO: 121 - DNA, 25 bp, Homo sapiens
gtgcctattaaacagagtactttgt

SEQ ID NO: 122 - DNA, 25 bp, Homo sapiens
ggaattcctatttctacgtatttgg

SEQ ID NO: 123 - DNA, 25 bp, Homo sapiens
gggcaccatatatcatctagagtcc

SEQ ID NO: 124 - DNA, 25 bp, Homo sapiens
atctagagtccttactggtattcag

SEQ ID NO: 125 - DNA, 25 bp, Homo sapiens
atgttcacgctgcattaattgtcat

SEQ ID NO: 126 - DNA, 25 bp, Homo sapiens
aaactagcttggaccttgggctgca

SEQ ID NO: 127 - DNA, 25 bp, Homo sapiens
gctgcaggacgactaggattcaccc

SEQ ID NO: 128 - DNA, 25 bp, Homo sapiens
ggattcacccataacgacacagtgc

SEQ ID NO: 129 - DNA, 25 bp, Homo sapiens
gacacagtgccctatgtttcttaac

SEQ ID NO: 130 - DNA, 25 bp, Homo sapiens
gtttcttaacttcttgttgccattt

SEQ ID NO: 131 - DNA, 25 bp, Homo sapiens
gccatttgaaactctgtactcttat

SEQ ID NO: 132 - DNA, 25 bp, Homo sapiens
taaagggttctgtatagccattttt

SEQ ID NO: 133 - DNA, 25 bp, Homo sapiens
gaagcaagtttccatgatttctgaa

SEQ ID NO: 134 - DNA, 25 bp, Homo sapiens
gaacgtttccaggcattttgtctcc

SEQ ID NO: 135 - DNA, 25 bp, Homo sapiens
ggagagttcacgaactgtgcccaac

SEQ ID NO: 136 - DNA, 25 bp, Homo sapiens
tgtgcccaacgcatgttatagccag

SEQ ID NO: 137 - DNA, 25 bp, Homo sapiens
tagccagggtcctactaactactca

SEQ ID NO: 138 - DNA, 25 bp, Homo sapiens
aacgtattgttgtattcctccagtg

SEQ ID NO: 139 - DNA, 25 bp, Homo sapiens
tcctccagtgttaagctatagccat

SEQ ID NO: 140 - DNA, 25 bp, Homo sapiens
cactgtgcatttattctcagcatca

SEQ ID NO: 141 - DNA, 25 bp, Homo sapiens
aaataccttgtaacgtcttctctgc

SEQ ID NO: 142 - DNA, 25 bp, Homo sapiens
tcttctctgccttgttagtgcatat

SEQ ID NO: 143 - DNA, 25 bp, Homo sapiens
ttctctgctccttaaatatcatctc

SEQ ID NO: 144 - DNA, 540 bp, Homo sapiens
aagctaggtcgtgggtcagctctgaggatgtatacccctggtgggagagggagacctagagatctggctgtggggcgggcatggggggtgaagggccactgggaccctcagccttgtttgtactgtatgccttcagcattgcctaggaacacgaagcacgatcagtccatccagagggaccggagttatgacaagcttcccaaatattttgctttatcagccgatatcaacacttgtatctggcctctgtgcccagcagtgccttgtgcaatgtgaatgtaccgtctctgctaaaccaccattttatttggttttgttttgtttggttttctcggatacttgccaaaatgagactctccgtcggcagctgggggaagggtctgagactctctttccttttggttttgggattacttttgatcctgggggaccaatgaggtgaggggggttctcctttgccctcagctttcccagccctccggcctgggctgcccacaaggcttctcccccagaggccctggctcctggtcgggaagggag

SEQ ID NO: 145 - DNA, 437 bp, Homo sapiens
tgcctgtagcattccagagctcactgcccttctagatgtgccttcccgcttggcttccagcggcttgtgctcactctgtctgccaggtatgagaagaacacgtaagaccgccaccacactcaccctccctcaaggccctgtgccataggggtggccacccgacctgcccccagaacttttggatactggaggcagttgcataggtctccctctctgggcaccaggactcagtccagcccaagactactctgggcagctcccatcccagtctggggccatttgcagactcaggaaaggatttctacagtgttctataaaagccaaaagagagagtgggtttgggaagagtgagggtggttggggagaggggaccgatgtgcctcattgtttagtggtgattacaaatatgcttttctggataaagtttggttgtttgc

SEQ ID NO: 146 - DNA, 514 bp, Homo sapiens
aatgcaggagcctacttctagctgtattttttgtatgcttaaataaaataaaattcataaccaagagatccacattagcttgttagtaatgctctgaccaagccgagatgccattctcttagtgatggcggcgttaggtttgagagaaggaattggctcaacttcagttgagagggtgcagtccagacagcttgactgcttttaaatgaccaaagatgacctgtggtaagcaacctggcatcttaggaagcagtccttgagaaggcatgttccagaaaggtctctgaggacaaactcactcagtaaaacataatgtatcatgaagaaaactgattctctatgacatgaaatgaaaattttaatgcattgttataattactaatgtacgctgctgcaggacattaataaagttgcttttttaggctacagtgtctcgatgccataatcagaacacactttttttcctctttctcccagcttcaaatgcacaattcatcattgggctcacttctaa

SEQ ID NO: 147 - DNA, 534 bp, Homo sapiens
aggtgcagaatctcggcggggagcttgttgtctctggggtggacagcgccatgtccctgatccaggcagccaagaacttgatgaatgctgtggtgcagacagtgaaggcatcctacgtcgcctctaccaaataccaaaagtcacagggtatggcttccctcaaccttcctgctgtgtcatggaagatgaaggcaccagagaaaaagccattggtgaagagagagaaacaggatgagacacagaccaagattaaacgggcatctcagaagaagcacgtgaacccggtgcaggccctcagcgagttcaaagctatggacagcatctaagtctgcccaggccggccgcccccacccctcggggctcctgaatatcagtcactgttcgtcactcaaatgaatttgctaaatacaacactgatactagattccacagggaaatgggcagactgaaccagtccaggtggtgaattttccaagaacatagtttaagttgattaaaaatgcttttagaatgcaggagcctacttctagctgt

SEQ ID NO: 148 - DNA, 540 bp, Homo sapiens
aatgcattgcatcaacctactatagaggagctcaagccatcatcattgtcttcaacctgaatgatgtggcatctctggaacataccaagcagtggctggccgatgccctgaaggagaatgacccttccagtgtgcttctcttccttgtaggttccaagaaggatctgagtacccctgctcagtatgcgctgatggagaaagacgccctccaggtggcccaggagatgaagactgttcagagactgcccagccctagggcactgtgccaccctcattcctccagagcttgacccctggacatttgcactgactttatccagaccaaagagctgcctcttggtggcagtattcccacagaggggtagctgggatcatgctagtcacttcctgcccccaggcaccgtgccaaagactggatgccccctactcctcaggggactgtccagggcgcccagtggtagtgagggagagtgtctctgttcttttgctcagcctgctgggccctttgtgtttgaggatgcttaatgattccagcctctc

SEQ ID NO: 149 - DNA, 331 bp, Homo sapiens
  misc_feature (113)..(113): n is a, c, g, or t
gagggagaactttcctgtggacgtcctgtgttctccagacgcagagaaccctcatcaaccgagggggaggtcacttccgaatccacagatggcgtgtgagtgcatggcgagcncctccaggacacacttactgttcccttgctctggccagacgccagccggaccctgtgtgtgcgcgccgtgctgctctttgcagctgcctgcaaggggttcctgcgaagaccagcaccttggggaagagcctgcggctgaacttgaactcgcagctacctgagtcagacctgtgctttttcacctctacggaagatgtcagagcgtttccctagcaatg

SEQ ID NO: 150 - DNA, 341 bp, Homo sapiens
cttttctcctgtgtaccaaggtattgcttttatttacacgacagcgactcaaaaggcactcgattaatgtgacaaccttttcaataagcagaaataacgtaggtacacatcactctttacatttttctaagcattttcacagccgtttcttcatataatccgaccacagtgggaggtgtgatttacccattacacaatgagaaaccagaggagccgatgagttacttaattgaggtcacagaatgaattagcaagaaaatggttctaaaatctaagtattttagtctagaattttctccattacatcatcctaagagataatgctctgtacttcatttgaa

SEQ ID NO: 151 - DNA, 389 bp, Homo sapiens
gatagtcggaatagagccgccccaactcagatcctacaacacgcaagttccttcttgaaccctggtgcctcctaccctatggccctgaatggtgcactggtttaattgtgttggtgtcggcccctcacaaatgcagccaagtcatgtaattagtcatctggaacaaagactaaaaacagcagagaattgcgggttctacccagtcagaagatcacaccatggagactttctactagaggacttgaaagagaactgaggggccacaaaataaacttcaccttccattaagtgttcaagcatgtctgcaaattaggagggagttagaaacagtctttttcatcctttgtgatgaagcctgaaattgtgccgtgttgccttatatgaatatg

SEQ ID NO: 152 - DNA, 503 bp, Homo sapiens
gagtacagacctgctgaatgcactgaagacagtaattaatgatccttcatataaagagaatgttatgaaattatcaataattcaacatgatcaaccagtaaagcccctgcatcgagcagtcttctggattgaatttgtgatgtgccacaaaggagccaaacaccttcgagttgcagcccgtgacctcacctggttccagtaccactctttggatgtgattgggtttctgctggcctgtgtggcaactgtgatatttgtcgtcacaaagttttgtctgttttgtttctggaagtttgctagaaaagggaagaagggaaaaagagattagttatgtctgacatttgaagctggaaaaccagatagatgggttgacatcagtttattccagcaagaaagaaaagattgttatgcaagatttctttcttcctgtgacaaaaaaaaaaacttttcaaaatctaccttgtcaagtaaaaatttgtttttcagagatttaccacccaggt

SEQ ID NO: 153 - DNA, 528 bp, Homo sapiens
caggaaacggcaacaagcctcccagtactgacctgaaaacttgtgacaagaagaacaccaacaagtgctccctgggctgaggaccctttcttgcctccccaccccggaagctgaacctgagggagacaacggcagagggagtgagcaggggagaaatagcagaggggcttggagggtcacataggtagatggtaaagagaatgaggagaaaaaggagaaaagggaaaagcagaaaggaaaaaaaggaagagagaggaagggagaagggagaggaatgaattgaggaagtgaaagaaggcaaggaggtaggaagagagggaggaggaaaggaaggagagagatgcctcaggcttcagaccttacctgggttttcagggcaaacataaatgtaaatacactgatttattctgttactagatcaggttttagggtcctgcaaaaggctagctcggcactacactagggaatttgctcctgttctgtcacttgtcatggtctttcttggtattaaaggccaccatttgcaca

SEQ ID NO: 154 - DNA, 408 bp, Homo sapiens
  misc_feature (317)..(317): n is a, c, g, or t
tctgctgcatttctaatgtgaattgcatgtgagagcttatgaatacactcttttcaagtgtatgtgggttttcaaggtatgtatttgtgtttgatcctatatttcctttgttataacattctgcttttcatcactttgacagaatatttgtaaattttctggagagtgagtttgggtatatgctttgaggaatatgcttcctcattacaaaagatatggaaacataattgattactacacttgcttatcaatagtattttgattttgtttaaatgaaatgtttaagatacgtggattgctatgcattcatgttttanatttatcctctccaggcatgatttatgccattcaataattaaaataactaagttactttagaaaaagtgcctattaaacagagtactttgt

SEQ ID NO: 155 - DNA, 489 bp, Homo sapiens
  misc_feature (68)..(68): n is a, c, g, or t
ggaattcctatttctacgtatttggtggtccataagactttgtcaaatgtaaacctacagtttgatangctttaaaatacctagttaagaggatgatttctctttaatcgtttaaatgttctgaaaattaaaatcttttgaggcacatgaagtgggcaccatatatcatctagagtccttactggtattcaggatgaaaatgttcacgctgcattaattgtcatttttctctcccatgttctttctcactttgatacgttaatactgataatggataaagagtgagtttttataataaatggttttggaaaggtattcataggaaccgcggttatttacttaaggttatggagtaaactagcttggaccttgggctgcaggacgactaggattcacccataacgacacagtgccctatgtttcttaacttcttgttgccatttgaaactctgtactcttatgtttaaagggttctgtatagccattttt

SEQ ID NO: 156 - DNA, 520 bp, Homo sapiens
gaagcaagtttccatgatttctgaagagctggtataggaagtttctttcttccttttgtgttacatgtgcattaaacagaacaagctgtgtgtcatcacagattgtactgtgggctcagaaaccgtgagagagcccccaccgtggacaccggctctatggccacaggaaaaggaacgtttccaggcattttgtctccagggctcccgctggacaggcacgtactgccccggggagtaaatgcggagagttcacgaactgtgcccaacgcatgttatagccagggtcctactaactactcagtaaaagaacgtattgttgtattcctccagtgttaagctatagccatgttaaaagtcactgtgcatttattctcagcatcaaataccttgtaacgtcttctctgccttgttagtgcatatttttacttttctgatactgtaaagaatatatccagtatgtaaatgaatgttctataaatcttttgtatagtcattttctctgctccttaaatatcatctc

SEQ ID NO: 157 - DNA, 20 bp, Homo sapiens
cgccatgccgggagaactct

SEQ ID NO: 158 - DNA, 20 bp, Homo sapiens
cttctcctgctgccggctgt

SEQ ID NO: 159 - DNA, 20 bp, Homo sapiens
gccgccttcaacgacgagtt

SEQ ID NO: 160 - DNA, 20 bp, Homo sapiens
cttggcttcatcctcctcgc

SEQ ID NO: 161 - DNA, 18 bp, Homo sapiens
cggccgctggtgatcaag

SEQ ID NO: 162 - DNA, 19 bp, Homo sapiens
cccagggcggtcccacagc